(19) United States
(12) Patent Application Publication
(10) Pub. No.: US 2021/0393775 A1
(43) Pub. Date: Dec. 23, 2021

(54) Daratumumab and Hyaluronidase for the Treatment of Multiple Myeloma

(71) Applicant: Janssen Biotech, Inc., Horsham, PA (US)

(72) Inventor: Ming Qi, Phoenixville, PA (US)

(21) Appl. No.: 17/343,150

(22) Filed: Jun. 9, 2021

Related U.S. Application Data
(60) Provisional application No. 63/038,438, filed on Jun. 12, 2020.

(51) Int. Cl. A61K 39/395; A61K 38/47; A61K 31/573; A61K 31/69; A61K 31/198; A61K 31/454; A61P 35/00

(52) U.S. Cl. CPC A61K 39/3955; A61K 38/47; A61K 31/573; A61K 2039/545; A61K 31/198; A61K 31/454; A61P 35/00; A61K 31/69

(57) ABSTRACT

The present invention relates to methods of treating multiple myeloma using an approved drug product comprising daratumumab and hyaluronidase. Also described are drug products containing daratumumab and hyaluronidase, and methods of selling or offering for sale a drug product comprising daratumumab and hyaluronidase.

### CROSS-REFERENCE TO RELATED APPLICATIONS

This application claims priority to U.S. Provisional Application Ser. No. 63/038,438, filed 12 Jun. 2020. The entire contents of the aforementioned application is incorporated herein by reference in its entirety.

### FIELD OF THE INVENTION

The present invention relates to methods of treating multiple myeloma using an approved drug product comprising daratumumab and hyaluronidase. Also described are drug products containing daratumumab and hyaluronidase, and methods of selling or offering for sale a drug product comprising daratumumab and hyaluronidase.

### BACKGROUND OF THE INVENTION

CD38 is a multifunctional protein having function in receptor-mediated adhesion and signaling as well as mediating calcium mobilization via its ecto-enzymatic activity, catalyzing formation of cyclic ADP-ribose (cADPR) and ADPR. CD38 mediates cytokine secretion and activation and proliferation of lymphocytes (Funaro et al., J Immunol 145:2390-6, 1990; Terhorst et al., Cell 771-80, 1981; Guse et al., Nature 398:70-3, 1999). CD38, via its NAD glycohydrolase activity, also regulates extracellular NAD⁺ levels, which have been implicated in modulating the regulatory T-cell compartment (Adriouch et al., 14:1284-92, 2012; Chiarugi et al., Nature Reviews 12:741-52, 2012). In addition to signaling via Ca²⁺, CD38 signaling occurs via cross-talk with antigen-receptor complexes on T- and B-cells or other types of receptor complexes, e.g., MHC molecules, involving CD38 in several cellular responses, but also in switching and secretion of IgG1. CD38 is expressed on various malignant cells.

Anti-CD38 antibodies are being developed for the treatment of multiple myeloma and other heme malignancies. The antibodies are either injected or infused via the intravenous (IV) route. The amount of antibody that can be administered via the intravenous route is limited by the physico-chemical properties of the antibody, in particularly by its solubility and stability in a suitable liquid formulation and by the volume of the infusion fluid.

Therefore, there is a need for additional anti-CD38 antibody formulations and pharmaceutical compositions.

### SUMMARY OF THE INVENTION

The invention provides a method of treating multiple myeloma comprising administering an approved drug product containing daratumumab and hyaluronidase to an adult patient with multiple myeloma in an amount that is described in a drug product label for the drug product.

The invention also provides a method of selling an approved drug product comprising daratumumab and hyaluronidase, the method comprising selling such drug product, wherein a drug product label for a reference listed drug for such drug product includes instructions for treating multiple myeloma.

The invention also provides a method of offering for sale an approved drug product comprising daratumumab and hyaluronidase, the method comprising offering for sale such drug product, wherein a drug product label for a reference listed drug for such drug product includes instructions for treating multiple myeloma.

The invention also provides a method of selling an approved drug product comprising daratumumab and hyaluronidase, the method comprising selling such drug product, wherein the drug product label for a reference listed drug for such drug product comprises ORR data.

The invention also provides a method of offering for sale an approved drug product comprising daratumumab and hyaluronidase, said method comprising offering for sale such drug product, wherein the drug product label for a reference listed drug for such drug product comprises ORR data.

The invention also provides a method of improving ORR in an adult patient with multiple myeloma, the method comprising administering to the adult patient an approved drug product comprising daratumumab and hyaluronidase.

### DETAILED DESCRIPTION OF THE INVENTION

“DARZALEX FASPRO” is a sterile, preservative-free, colorless to yellow, and clear to opalescent solution for subcutaneous injection use supplied as individually packaged single-dose vials providing 1,800 mg of daratumumab and 30,000 units of hyaluronidase per 15 mL.

The term, “drug product” or “approved drug product” is product that contains an active pharmaceutical ingredient that has been approved for marketing for at least one indication by a governmental authority, e.g., the Food and Drug Administration or the similar authority in other countries

“CD38” refers to the human CD38 protein (synonyms: ADP-ribosyl cyclase 1, cADPr hydrolase 1, cyclic ADP-ribose hydrolase 1). Human CD38 has an amino acid sequence shown in GenBank accession number NP_001766 and in SEQ ID NO: 1. It is well known that CD38 is a single pass type II membrane protein with amino acid residues 1-21 representing the cytosolic domain, amino acid residues 22-42 representing the transmembrane domain, and residues 43-300 representing the extracellular domain of CD38.

SEQ ID NO: 1
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI

“Antibodies” is meant in a broad sense and includes immunoglobulin molecules including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy (H) chains and two light (L) chains inter-connected by disulfide bonds as well as multimers thereof (for example IgM). Each heavy chain is comprised of a heavy chain variable region (VH) and a heavy chain constant region (comprised of domains CH1, hinge CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-terminus to carboxy-terminus in the following order: FR1, CDR1, FR2, CDR2, FR3, CDR3, and FR4.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, J Exp Med 132:211-50, 1970; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, Mol Biol 196:901-17, 1987). The International ImMunoGeneTics (IMGT) database (http://www.imgt.org) provides a standardized numbering and definition of antigen-binding sites. The correspondence between CDRs, HVs and IMGT delineations is described in Lefranc et al., Dev Comparat Immunol 27:55-77, 2003. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄. Antibody light chains of any vertebrate species can be assigned to one of two clearly distinct types, namely kappa (κ) and lambda (2), based on the amino acid sequences of their constant domains.

“Antigen-binding fragment” refers to a portion of an immunoglobulin molecule that retains the antigen binding properties of the parental full length antibody. Exemplary antigen-binding fragments are as heavy chain complementarity determining regions (HCDR) 1, 2 and/or 3, light chain complementarity determining regions (LCDR) 1, 2 and/or 3, a heavy chain variable region (VH), or a light chain variable region (VL), Fab, F(ab′)2, Fd and Fv fragments as well as domain antibodies (dAb) consisting of either one VH domain or one VL domain. VH and VL domains may be linked together via a synthetic linker to form various types of single chain antibody designs in which the VH/VL domains pair intramolecularly, or intermolecularly in those cases when the VH and VL domains are expressed by separate chains, to form a monovalent antigen binding site, such as single chain Fv (scFv) or diabody; described for example in Int. Pat. Publ. No. WO1998/44001, Int. Pat. Publ. No. WO1988/01649; Int. Pat. Publ. No. WO1994/13804; Int. Pat. Publ. No. WO1992/01047.

“Monoclonal antibody” refers to an antibody population with single amino acid composition in each heavy and each light chain, except for possible well known alterations such as removal of C-terminal lysine from the antibody heavy chain. Monoclonal antibodies typically bind one antigenic epitope, except that multispecific monoclonal antibodies bind two or more distinct antigens or epitopes. Bispecific monoclonal antibodies bind two distinct antigenic epitopes. Monoclonal antibodies may have heterogeneous glycosylation within the antibody population. Monoclonal antibody may be monospecific or multispecific, or monovalent, bivalent or multivalent. A multispecific antibody, such as a bispecific antibody or a trispecific antibody is included in the term monoclonal antibody.

“Isolated antibody” refers to an antibody or an antigen-binding fragment thereof that is substantially free of other antibodies having different antigenic specificities (e.g., an isolated antibody specifically binding human CD38 is substantially free of antibodies that specifically bind antigens other than human CD38). In case of a bispecific antibody, the bispecific antibody specifically binds two antigens of interest, and is substantially free of antibodies that specifically bind antigens other that the two antigens of interest. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibodies” refers to antibodies in which the antigen binding sites are derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibodies” refers to antibodies having heavy and light chain variable regions in which both the framework and the antigen binding site are derived from sequences of human origin. If the antibody contains a constant region or a portion of the constant region, the constant region also is derived from sequences of human origin.

A human antibody comprises heavy or light chain variable regions that are derived from sequences of human origin if the variable regions of the antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci as described herein. A human antibody typically contains amino acid differences when compared to the human germline or rearranged immunoglobulin sequences due to, for example naturally occurring somatic mutations, intentional introduction of substitutions into the framework or antigen binding site and amino acid changes introduced during cloning and VDJ recombination in non-human animals. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene. In some cases, a human antibody may contain consensus framework sequences derived from human framework sequence analyses, for example as described in Knappik et al., J Mol Biol 296:57-86, 2000, or synthetic HCDR3 incorporated into human immunoglobulin gene libraries displayed on phage, for example as described in Shi et al., J Mol Biol 397:385-96, 2010 and Int. Pat. Publ. No. WO2009/085462.

Antibodies in which antigen binding sites are derived from a non-human species are not included in the definition of human antibody.

“Recombinant” includes antibodies and other proteins that are prepared, expressed, created or isolated by recombinant means.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Multispecific” refers to an antibody that specifically binds at least two distinct antigens or two distinct epitopes within the antigens, for example three, four or five distinct antigens or epitopes.

“Bispecific” refers to an antibody that specifically binds two distinct antigens or two distinct epitopes within the same antigen. The bispecific antibody may have cross-reactivity to other related antigens or can bind an epitope that is shared between two or more distinct antigens.

“Variant” refers to a polypeptide or a polynucleotide that differs from a reference polypeptide or a reference polynucleotide by one or more modifications for example, substitutions, insertions or deletions.

“In combination with” means that two or more therapeutics are administered to a subject together in a mixture, concurrently as single agents or sequentially as single agents in any order.

“Pharmaceutical composition” refers to a product that results from combining an anti-CD38 antibody and a hyaluronidase and includes both fixed and non-fixed combinations. Pharmaceutical composition typically includes a pharmaceutically acceptable carrier. “Fixed combinations” refers to a single pharmaceutical composition comprising the anti-CD38 antibody and the hyaluronidase administered simultaneously in the form of a single entity or dosage. “Non-fixed combination” refers to separate pharmaceutical compositions of the anti-CD38 antibody and the hyaluronidase or unit dosage forms administered as separate entities either simultaneously, concurrently or sequentially with no specific intervening time limits, wherein such administration provides effective levels of the two compounds in the body of the subject.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

“Treat” or “treatment” refers to therapeutic treatment wherein the object is to slow down (lessen) an undesired physiological change or disease, such as the development or spread of tumor or tumor cells, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, lack of metastasis, amelioration or palliation of the disease state, and remission (whether partial or total), whether detectable or undetectable. “Treatment” may also mean prolonging survival as compared to expected survival if a subject was not receiving treatment. Those in need of treatment include those subjects already with the undesired physiological change or disease well as those subjects prone to have the physiological change or disease.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body.

“Inhibits growth” (e.g. referring to tumor cells) refers to a measurable decrease in the tumor cell growth or tumor tissue in vitro or in vivo when contacted with a therapeutic or a combination of therapeutics or drugs, when compared to the growth of the same tumor cells or tumor tissue in the absence of the therapeutic or the combination of therapeutic drugs. Inhibition of growth of a tumor cell or tumor tissue in vitro or in vivo may be at least about 10%, 20%, 30%, 40%, 50%, 60%, 70%, 80%, 90%, 99%, or 100%.

“CD38-positive hematological malignancy” refers to a hematological malignancy characterized by the presence of tumor cells expressing CD38 including leukemias, lymphomas and myeloma. Examples of such CD38-positive hematological malignancies include precursor B-cell lymphoblastic leukemia/lymphoma and B-cell non-Hodgkin's lymphoma, acute promyelocytic leukemia, acute lymphoblastic leukemia and mature B-cell neoplasms, such as B-cell chronic lymphocytic leukemia (CLL)/small lymphocytic lymphoma (SLL), B-cell acute lymphocytic leukemia, B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma (BL), plasmacytoma, multiple myeloma, plasma cell leukemia, post-transplant lymphoproliferative disorder, light chain amyloidosis, Waldenstrom's macroglobulinemia, plasma cell leukemias and anaplastic large-cell lymphoma (ALCL).

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

### Pharmaceutical Compositions

The invention provides a pharmaceutical composition comprising an anti-CD38 antibody and a hyaluronidase.

The pharmaceutical composition is useful for subcutaneous administration of the anti-CD38 antibody to a subject in need of anti-CD38 antibody therapy, such as a subject having a cancer, for example a CD38-positive hematological malignancy. Without wishing to be bound by any particular theory, subcutaneous administration of the anti-CD38 antibody may have reduced infusion related reaction and achieve improved response rates when compared to the intravenous administration of the anti-CD38 antibody.

In some embodiments, the pharmaceutical composition is a fixed combination.

In some embodiments, the pharmaceutical composition is a non-fixed combination.

In some embodiments, the pharmaceutical composition comprises from about 1 mg/mL to about 180 mg/mL of the anti-CD38 antibody.

In some embodiments, the pharmaceutical composition comprises from about 10 mg/mL to about 180 mg/mL of the anti-CD38 antibody.

In some embodiments, the pharmaceutical composition comprises from about 20 mg/mL to about 160 mg/mL of the anti-CD38 antibody.

In some embodiments, the pharmaceutical composition comprises from about 20 mg/mL to about 140 mg/mL of the anti-CD38 antibody.

In some embodiments, the pharmaceutical composition comprises from about 20 mg/mL to about 120 mg/mL of the anti-CD38 antibody.

In some embodiments, the pharmaceutical composition comprises from about 40 mg/mL to about 120 mg/mL of the anti-CD38 antibody.

In some embodiments, the pharmaceutical composition comprises from about 60 mg/mL to about 120 mg/mL of the anti-CD38 antibody.

In some embodiments, the pharmaceutical composition comprises from about 80 mg/mL to about 120 mg/mL of the anti-CD38 antibody.

In some embodiments, the pharmaceutical composition comprises from about 100 mg/mL to about 120 mg/mL of the anti-CD38 antibody.

In some embodiments, the pharmaceutical composition comprises about 1 mg/mL, about 5 mg/mL, about 10 mg/mL, about 15 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL or about 180 mg/mL of the anti-CD38 antibody.

In some embodiments, the pharmaceutical composition comprises about 20 mg/mL of the anti-CD38 antibody.

In some embodiments, the pharmaceutical composition comprises about 100 mg/mL of the anti-CD38 antibody.

In some embodiments, the pharmaceutical composition comprises about 120 mg/mL of the anti-CD38 antibody.

In some embodiments, the pharmaceutical composition comprises from about 50 U/mL to about 5,000 U/mL of the hyaluronidase.

In some embodiments, the pharmaceutical composition comprises from about 500 U/mL to about 5,000 U/mL of the hyaluronidase.

In some embodiments, the pharmaceutical composition comprises from about 1,000 U/mL to about 5,000 U/mL of the hyaluronidase.

In some embodiments, the pharmaceutical composition comprises from about 2,000 U/mL to about 5,000 U/mL of the hyaluronidase.

In some embodiments, the pharmaceutical composition comprises from about 50 U/mL to about 2,000 U/mL of the hyaluronidase.

In some embodiments, the pharmaceutical composition comprises from about 500 U/mL to about 2,000 U/mL of the hyaluronidase.

In some embodiments, the pharmaceutical composition comprises from about 1,000 U/mL to about 2,000 U/mL of the hyaluronidase.

In some embodiments, the pharmaceutical composition comprises about 500 U/mL, about 600 U/mL, about 700 U/mL, about 800 U/mL, about 900 U/mL, about 1,000 U/mL, about 1,100 U/mL, about 1,200 U/mL, about 1,300 U/mL, about 1,400 U/mL, about 1,500 U/mL, about 1,600 U/mL, about 1,700 U/mL, about 1,800 U/mL, about 1,900 U/mL, about 2,000 U/mL, about 2,100 U/mL, about 2,200 U/mL, about 2,300 U/mL, about 2,400 U/mL, about 2,500 U/mL, about 2,600 U/mL, about 2,700 U/mL, about 2,800 U/mL, about 2,900 U/mL, about 3,000 U/mL, about 3,100 U/mL, about 3,200 U/mL, about 3,300 U/mL, about 3,400 U/mL, about 3,500 U/mL, about 3,600 U/mL, about 3,700 U/mL, about 3,800 U/mL, about 3,900 U/mL, about 4,000 U/mL, about 4,100 U/mL, about 4,200 U/mL, about 4,300 U/mL, about 4,400 U/mL, about 4,500 U/mL, about 4,600 U/mL, about 4,700 U/mL, about 4,800 U/mL, about 4,900 U/mL or about 5,000 U/mL of the hyaluronidase.

In some embodiments, the pharmaceutical composition comprises about 500 U/mL of the hyaluronidase.

In some embodiments, the pharmaceutical composition comprises about 2,000 U/mL of the hyaluronidase.

In some embodiments, the pharmaceutical composition comprises about 5,000 U/mL of the hyaluronidase.

In some embodiments, the pharmaceutical composition comprises from about 1,200 mg to about 5,000 mg of the anti-CD38 antibody.

In some embodiments, the pharmaceutical composition comprises from about 1,200 mg to about 2,400 mg of the anti-CD38 antibody.

In some embodiments, the pharmaceutical composition comprises from about 1,200 mg to about 1,800 mg of the anti-CD38 antibody.

In some embodiments, the pharmaceutical composition comprises about 1,200 mg of the anti-CD38 antibody.

In some embodiments, the pharmaceutical composition comprises about 1,400 mg of the anti-CD38 antibody.

In some embodiments, the pharmaceutical composition comprises about 1,600 mg of the anti-CD38 antibody.

In some embodiments, the pharmaceutical composition comprises about 1,800 mg of the anti-CD38 antibody.

In some embodiments, the pharmaceutical composition comprises about 2,000 mg of the anti-CD38 antibody.

In some embodiments, the pharmaceutical composition comprises about 2,200 mg of the anti-CD38 antibody.

In some embodiments, the pharmaceutical composition comprises about 2,400 mg of the anti-CD38 antibody.

In some embodiments, the pharmaceutical composition comprises about 2,600 mg of the anti-CD38 antibody.

In some embodiments, the pharmaceutical composition comprises about 2,800 mg of the anti-CD38 antibody.

In some embodiments, the pharmaceutical composition comprises about 3,000 mg of the anti-CD38 antibody.

In some embodiments, the pharmaceutical composition comprises about 3,500 mg of the anti-CD38 antibody.

In some embodiments, the pharmaceutical composition comprises about 4,000 mg of the anti-CD38 antibody.

In some embodiments, the pharmaceutical composition comprises about 4,500 mg of the anti-CD38 antibody.

In some embodiments, the pharmaceutical composition comprises about 5,000 mg of the anti-CD38 antibody.

In some embodiments, the pharmaceutical composition comprises from about 750 U to about 75,000 U of the hyaluronidase.

In some embodiments, the pharmaceutical composition comprises from about 7,500 U to about 45,000 U of the hyaluronidase.

In some embodiments, the pharmaceutical composition comprises from about 30,000 U to about 45,000 U of the hyaluronidase.

In some embodiments, the pharmaceutical composition comprises about 7,500 U, about 8,000 U, about 8,500 U, about 9,000 U, about 10,000 U, about 15,000 U, about 20,000 U, about 21,000 U, about 22,000 U, about 23,000 U, about 24,000 U, about 25,000 U, about 26,000 U, about 27,000 U, about 28,000 U, about 29,000 U, about 30,000 U, about 31,000 U, about 32,000 U, about 33,000 U, about 34,000 U, about 35,000 U, about 36,000 U, about 37,000 U, about 38,000 U, about 39,000 U, about 40,000 U, about 41,000 U, about 42,000 U, about 43,000 U, about 44,000 U, about 45,000 U, about 46,000 U, about 47,000 U, about 48,000 U, about 49,000 U, about 50,000 U, about 55,000 U, about 60,000 U, about 65,000 U, about 70,000 U or about 75,000 U of the hyaluronidase.

In some embodiments, the pharmaceutical composition comprises about 5,000 mg of the anti-CD38 antibody and about 30,000 U of the hyaluronidase.

In some embodiments, the pharmaceutical composition comprises about 5,000 mg of the anti-CD38 antibody and about 45,000 U of the hyaluronidase.

In some embodiments, the pharmaceutical composition comprises about 3,000 mg of the anti-CD38 antibody and about 30,000 U of the hyaluronidase.

In some embodiments, the pharmaceutical composition comprises about 3,000 mg of the anti-CD38 antibody and about 45,000 U of the hyaluronidase.

In some embodiments, the pharmaceutical composition comprises about 2,800 mg of the anti-CD38 antibody and about 30,000 U of the hyaluronidase.

In some embodiments, the pharmaceutical composition comprises about 2,800 mg of the anti-CD38 antibody and about 45,000 U of the hyaluronidase.

In some embodiments, the pharmaceutical composition comprises about 2,600 mg of the anti-CD38 antibody and about 30,000 U of the hyaluronidase.

In some embodiments, the pharmaceutical composition comprises about 2,600 mg of the anti-CD38 antibody and about 45,000 U of the hyaluronidase.

In some embodiments, the pharmaceutical composition comprises about 2,400 mg of the anti-CD38 antibody and about 30,000 U of the hyaluronidase.

In some embodiments, the pharmaceutical composition comprises about 2,400 mg of the anti-CD38 antibody and about 45,000 U of the hyaluronidase.

In some embodiments, the pharmaceutical composition comprises about 2,200 mg of the anti-CD38 antibody and about 30,000 U of the hyaluronidase.

In some embodiments, the pharmaceutical composition comprises about 2,200 mg of the anti-CD38 antibody and about 45,000 U of the hyaluronidase.

In some embodiments, the pharmaceutical composition comprises about 2,000 mg of the anti-CD38 antibody and about 30,000 U of the hyaluronidase.

In some embodiments, the pharmaceutical composition comprises about 2,000 mg of the anti-CD38 antibody and about 45,000 U of the hyaluronidase.

In some embodiments, the pharmaceutical composition comprises about 1,800 mg of the anti-CD38 antibody and about 30,000 U of the hyaluronidase.

In some embodiments, the pharmaceutical composition comprises about 1,800 mg of the anti-CD38 antibody and about 45,000 U of the hyaluronidase.

In some embodiments, the pharmaceutical composition comprises about 1,600 mg of the anti-CD38 antibody and about 30,000 U of the hyaluronidase.

In some embodiments, the pharmaceutical composition comprises about 1,600 mg of the anti-CD38 antibody and about 45,000 U of the hyaluronidase.

In some embodiments, the hyaluronidase is rHuPH20 having the amino acid sequence of SEQ ID NO: 22.

rHuPH20 is a recombinant hyaluronidase (HYLENEX® recombinant) and is described in Int. Pat. Publ. No. WO2004/078140.

Hyaluronidase is an enzyme that degrades hyaluronic acid (EC 3.2.1.35) and lowers the viscosity of hyaluronan in the extracellular matrix, thereby increasing tissue permeability.

Enzymatic activity of hyaluronidase, including rHuPH20 can be defined by units per mL (U/mL) or by total enzyme activity in a particular formulation (U).

The standard definition for one unit (U) of enzyme activity is the amount of enzyme that catalyzes the reaction of 1 nmol of substrate per minute.

In some embodiments, the anti-CD38 antibody in the pharmaceutical composition competes for binding to CD38 with an antibody comprising a heavy chain variable region (VH) of SEQ ID NO: 4 and a light chain variable region (VL) of SEQ ID NO: 5.

In some embodiments, the anti-CD38 antibody in the pharmaceutical composition binds at least to the region SKRNIQFSCKNIYR (SEQ ID NO: 2) and the region EKVQTLEAWVIHGG (SEQ ID NO: 3) of human CD38 (SEQ ID NO: 1).

In some embodiments, the anti-CD38 antibody in the pharmaceutical composition comprises a heavy chain complementarity determining region 1 (HCDR1), a HCDR2, a HCDR3, a light chain complementarity determining region 1 (LCDR1), a LCDR2 and a LCDR3 of SEQ ID NOs: 6, 7 and 8, 9, 10 and 11, respectively.

In some embodiments, the anti-CD38 antibody in the pharmaceutical composition comprises a heavy chain variable region (VH) that is 95%, 96%, 97%, 98%, 99% or 100% identical to the amino acid sequence of SEQ ID NO: 4 and a light chain variable region (VL) that is 95%, 96%, 97%, 98%, 99% or 100% identical to the amino acid sequence of SEQ ID NO: 5.

In some embodiments, the anti-CD38 antibody in the pharmaceutical composition comprises the VH of SEQ ID NO: 4 and the VL of SEQ ID NO: 5.

In some embodiments, the anti-CD38 antibody in the pharmaceutical composition comprises a heavy chain of SEQ ID NO: 12 and a light chain of SEQ ID NO: 13.

SEQ ID NO: 2
SKRNIQFSCKNIYR
SEQ ID NO: 3
EKVQTLEAWVIHGG
SEQ ID NO: 4
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSS
SEQ ID NO: 5
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIK
SEQ ID NO: 6
SFAMS
SEQ ID NO: 7
AISGSGGGTYYADSVKG
SEQ ID NO: 8
DKILWFGEPVFDY
SEQ ID NO: 9
RASQSVSSYLA
SEQ ID NO: 10
DASNRAT
SEQ ID NO: 11
QQRSNWPPTF
SEQ ID NO: 12
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 13
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Other exemplary anti-CD38 antibodies that may be used in the pharmaceutical compositions and the methods of the invention are:

mAb003 comprising the VH and the VL sequences of SEQ ID NOs: 14 and 15, respectively and described in U.S. Pat. No. 7,829,693. The VH and the VL of mAb003 may be expressed as IgG1/K;

mAb024 comprising the VH and the VL sequences of SEQ ID NOs: 16 and 17, respectively, described in U.S. Pat. No. 7,829,693. The VH and the VL of mAb024 may be expressed as IgG1/K;

MOR-202 (MOR-03087) comprising the VH and the VL sequences of SEQ ID NOs: 18 and 19, respectively, described in U.S. Pat. No. 8,088,896. The VH and the VL of MOR-202 may be expressed as IgG1/κ; or

Isatuximab; comprising the VH and the VL sequences of SEQ ID NOs: 20 and 21, respectively, described in U.S. Pat. No. 8,153,765. The VH and the VL of Isatuximab may be expressed as Ig

SEQ ID NO: 14
QVQLVQSGAEVKKPGSSVKVSCKASGGTFSSYAFSWVRQAPGQGLEWMGR
VIPFLGIANSAQKFQGRVTITADKSTSTAYMDLSSLRSEDTAVYYCARDD
IAALGPFDYWGQGTLVTVSSAS
SEQ ID NO: 15
DIQMTQSPSSLSASVGDRVTITCRASQGISSWLAWYQQKPEKAPKSLIYA
ASSLQSGVPSRFSGSGSGTDFTLTISSLQPEDFATYYCQQYNSYPRTFGQ
GTKVEIK
SEQ ID NO: 16
EVQLVQSGAEVKKPGESLKISCKGSGYSFSNYWIGWVRQMPGKGLEWMGI
IYPHDSDARYSPSFQGQVTFSADKSISTAYLQWSSLKASDTAMYYCARHV
GWGSRYWYFDLWGRGTLVTVSS
SEQ ID NO: 17
EIVLTQSPATLSLSPGERATLSCRASQSVSSYLAWYQQKPGQAPGLLIYD
ASNRASGIPARFSGSGSGTDFTLTISSLEPEDFAVYYCQQRSNWPLTFGG
GTKVEIK
SEQ ID NO: 18
QVQLVESGGGLVQPGGSLRLSCAASGFTFSSYYMNWVRQAPGKGLEWVSG
ISGDPSNTYYADSVKGRFTISRDNSKNTLYLQMNSLRAEDTAVYYCARDL
PLVYTGFAYWGQGTLVTVSS
SEQ ID NO: 19
DIELTQPPSVSVAPGQTARISCSGDNLRHYYVYWYQQKPGQAPVLVIYGD
SKRPSGIPERFSGSNSGNTATLTISGTQAEDEADYYCQTYTGGASLVFGG
GTKLTVLGQ
SEQ ID NO 20:
QVQLVQSGAEVAKPGTSVKLSCKASGYTFTDYWMQWVKQRPGQGLEWIGT
IYPGDGDTGYAQKFQGKATLTADKSSKTVYMHLSSLASEDSAVYYCARGD
YYGSNSLDYWGQGTSVTVSS
SEQ ID NO: 21:
DIVMTQSHLSMSTSLGDPVSITCKASQDVSTVVAWYQQKPGQSPRRLIYS
ASYRYIGVPDRFTGSGAGTDFTFTISSVQAEDLAVYYCQQHYSPPYTFGG
GTKLEIK

Other exemplary anti-CD38 antibodies that may be used in the pharmaceutical compositions of the invention are those described in Int. Pat. Publ. No. WO05/103083, Intl. Pat. Publ. No. WO06/125640, Intl. Pat. Publ. No. WO07/042309, Intl. Pat. Publ. No. WO08/047242 or Intl. Pat. Publ. No. WO14/178820.

An exemplary anti-CD38 antibody that may be used in the pharmaceutical compositions of the invention is daratumumab. Daratumumab comprises the heavy chain variable region (VH) and the light chain variable region (VL) amino acid sequences shown in SEQ ID NOs: 4 and 5, respectively, the HCDR1, the HCDR2 and the HCDR3 of SEQ ID NOs: 6, 7 and 8, respectively, the LCDR1, the LCDR2 and the LCDR3 of SEQ ID NOs: 9, 10 and 11, respectively, and is of IgG1/κ subtype and described in U.S. Pat. No. 7,829,693. Daratumumab heavy chain amino acid sequence is shown in SEQ ID NO: 12 and light chain amino acid sequence shown in SEQ ID NO: 13.

The invention also provides a pharmaceutical composition comprising an anti-CD38 antibody comprising the VH of SEQ ID NO: 4 and the VL of SEQ ID NO: 5 and a hyaluronidase rHuPH20 of SEQ ID NO: 22, wherein the anti-CD38 antibody concentration in the pharmaceutical composition is about 20 mg/mL.

The invention also provides a pharmaceutical composition comprising an anti-CD38 antibody comprising the HCDR1, the HCDR2 and the HCDR3 of SEQ ID NOs: 6, 7 and 8, respectively, and the LCDR1, the LCDR2 and the LCDR3 of SEQ ID NOs: 9, 10 and 11, respectively and the hyaluronidase rHuPH20 of SEQ ID NO: 22, wherein the anti-CD38 antibody concentration in the pharmaceutical composition is about 20 mg/mL.

The invention also provides a pharmaceutical composition comprising between about 1,200 mg-1,800 mg of the anti-CD38 antibody comprising the VH of SEQ ID NO: 4 and the VL of SEQ ID NO: 5, and between about 30,000 U-45,000 U of the hyaluronidase rHuPH20 of SEQ ID NO: 22, wherein the anti-CD38 antibody concentration in the pharmaceutical composition is about 20 mg/mL.

The invention also provides a pharmaceutical composition comprising about 1,800 mg of the anti-CD38 antibody comprising the VH of SEQ ID NO: 4 and the VL of SEQ ID NO: 5, and about 30,000 U of the hyaluronidase rHuPH20 of SEQ ID NO: 22, wherein the anti-CD38 antibody concentration in the pharmaceutical composition is about 20 mg/mL.

The invention also provides a pharmaceutical composition comprising about 1,800 mg of the anti-CD38 antibody comprising the VH of SEQ ID NO: 4 and the VL of SEQ ID NO: 5, and about 45,000 U of the hyaluronidase rHuPH20 of SEQ ID NO: 22, wherein the anti-CD38 antibody concentration in the pharmaceutical composition is about 20 mg/mL.

The invention also provides a pharmaceutical composition comprising about 1,600 mg of the anti-CD38 antibody comprising the VH of SEQ ID NO: 4 and the VL of SEQ ID NO: 5, and about 30,000 U of the hyaluronidase rHuPH20 of SEQ ID NO: 22, wherein the anti-CD38 antibody concentration in the pharmaceutical composition is about 20 mg/mL.

The invention also provides a pharmaceutical composition comprising about 1,600 mg of the anti-CD38 antibody comprising the VH of SEQ ID NO: 4 and the VL of SEQ ID NO: 5, and about 45,000 U of the hyaluronidase rHuPH20 of SEQ ID NO: 22, wherein the anti-CD38 antibody concentration in the pharmaceutical composition is about 20 mg/mL.

The invention also provides a pharmaceutical composition comprising about 1,200 mg of the anti-CD38 antibody comprising the VH of SEQ ID NO: 4 and the VL of SEQ ID NO: 5, and about 30,000 U of the hyaluronidase rHuPH20 of SEQ ID NO: 22, wherein the anti-CD38 antibody concentration in the pharmaceutical composition is about 20 mg/mL.

The invention also provides a pharmaceutical composition comprising about 1,200 mg of the anti-CD38 antibody comprising the VH of SEQ ID NO: 4 and the VL of SEQ ID NO: 5, and about 45,000 U of the hyaluronidase rHuPH20 of SEQ ID NO: 22, wherein the anti-CD38 antibody concentration in the pharmaceutical composition is about 20 mg/mL.

SEQ ID NO: 22
MGVLKFKHIFFRSFVKSSGVSQIVFTFLLIPCCLTLNFRAPPVIPNVPFL
WAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEW
RPTWARNWKPKDVYKNRSIELVQQQNVQLSLTEATEKAKQEFEKAGKDFL
VETIKLGKLLRPNHLWGYYLFPDCYNHHYKKPGYNGSCFNVEIKRNDDLS
WLWNESTALYPSIYLNTQQSPVAATLYVRNRVREAIRVSKIPDAKSPLPV
FAYTRIVFTDQVLKFLSQDELVYTFGETVALGASGIVIWGTLSIMRSMKS
CLLLDNYMETILNPYIINVTLAAKMCSQVLCQEQGVCIRKNWNSSDYLHL
NPDNFAIQLEKGGKFTVRGKPTLEDLEQFSEKFYCSCYSTLSCKEKADVK
DTDAVDVCIADGVCIDAFLKPPMETEEPQIFYNASPSTLSATMFIVSILF
LIISSVASL

Anti-CD38 antibodies used in the pharmaceutical compositions of the invention, may also be selected de novo from, e.g., a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-314, 1996; Sheets et al., PITAS (USA) 95:6157-6162, 1998; Hoogenboom and Winter, J Mol Biol 227:381, 1991; Marks et al., J Mol Biol 222:581, 1991). CD38 binding variable domains may be isolated from e.g., phage display libraries expressing antibody heavy and light chain variable regions as fusion proteins with bacteriophage pIX coat protein as described in Shi et al., J. Mol. Biol. 397:385-96, 2010 and Intl. Pat. Publ. No. WO09/085462). The antibody libraries may be screened for binding to human CD38 extracellular domain, the obtained positive clones further characterized, Fabs isolated from the clone lysates, and subsequently cloned as full length antibodies. Such phage display methods for isolating human antibodies are established in the art. See for example: U.S. Pat. Nos. 5,223,409, 5,403,484, 5,571,698, 5,427,908, 5,580,717, 5,969,108, 6,172,197, 5,885,793, 6,521,404, 6,544,731, 6,555,313, 6,582,915, and 6,593,081.

Antibodies may be evaluated for their competition with a reference antibody such as the anti-CD38 antibody comprising the VH of SEQ ID NO: 4 and the VL of SEQ ID NO: 5 for binding to CD38 using known in vitro methods. In an exemplary method, CHO cells recombinantly expressing CD38 may be incubated with unlabeled reference antibody for 15 min at 4° C., followed by incubation with an excess of fluorescently labeled test antibody for 45 min at 4° C. After washing in PBS/BSA, fluorescence may be measured by flow cytometry using standard methods. In another exemplary method, extracellular portion of human CD38 may be coated on the surface of an ELISA plate. Excess of unlabeled reference antibody may be added for about 15 minutes and subsequently biotinylated test antibodies may be added. After washes in PBS/Tween, binding of the test biotinylated antibody may be detected using horseradish peroxidase (HRP)-conjugated streptavidine and the signal detected using standard methods. It is readily apparent that in the competition assays, the reference antibody may be labelled and the test antibody unlabeled. The test antibody competes with the reference antibody when the reference antibody inhibits binding of the test antibody, or the test antibody inhibits binding of the reference antibody by at least 80%, for example 81%, 82%, 83%, 84%, 85%, 86%, 87%, 88%, 89%, 90%, 91%, 92%, 93%, 94%, 95%, 96%, 97%, 98%, 99% or 100%. The epitope of the test antibody may further be defined for example by peptide mapping or hydrogen/deuterium protection assays using known methods, or by crystal structure determination.

Antibodies binding to the region SKRNIQFSCKNIYR (SEQ ID NO: 2) and the region EKVQTLEAWVIHGG (SEQ ID NO: 3) of human CD38 (SEQ ID NO: 1) may be generated for example by immunizing mice with peptides having the amino acid sequences shown in SEQ ID NOs: 2 and 3 using standard methods and those described herein, and characterizing the obtained antibodies for binding to the peptides using for example ELISA or mutagenesis studies.

The invention also provides a pharmaceutical composition comprising an anti-CD38 antibody comprising the HCDR1, the HCDR2, the HCDR3, the LCDR1, the LCDR2, and the LCDR3 sequences of:
a. the VH of SEQ ID NO: 14 and the VL of SEQ ID NO: 15;
b. the VH of SEQ ID NO: 16 and the VL of SEQ ID NO: 17;
c. the VH of SEQ ID NO: 18 and the VL of SEQ ID NO: 19; or
d. the VH of SEQ ID NO: 20 and the VL of SEQ ID NO: 21, and the hyaluronidase rHuPH20 of SEQ ID NO: 22.

The invention also provides a pharmaceutical composition comprising an anti-CD38 antibody comprising
a. the VH of SEQ ID NO: 14 and the VL of SEQ ID NO: 15;
b. the VH of SEQ ID NO: 16 and the VL of SEQ ID NO: 17;
c. the VH of SEQ ID NO: 18 and the VL of SEQ ID NO: 19; or
d. the VH of SEQ ID NO: 20 and the VL of SEQ ID NO: 21, and the hyaluronidase rHuPH20 of SEQ ID NO: 22.

The pharmaceutical compositions of the invention further comprise a pharmaceutically acceptable carrier. Exemplary pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Exemplary buffers that may be used are acetic acid, citric acid, formic acid, succinic acid, phosphoric acid, carbonic acid, malic acid, aspartic acid, histidine, boric acid, Tris buffers, HEPPSO and HEPES.

Exemplary antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Exemplary amino acids that may be used are histidine, isoleucine, methionine, glycine, arginine, lysine, L-leucine, tri-leucine, alanine, glutamic acid, L-threonine, and 2-phenylamine.

Exemplary surfactants that may be used are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc).

Exemplary preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Exemplary saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Exemplary salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. An exemplary salt is sodium chloride.

The amounts of pharmaceutically acceptable carrier(s) in the pharmaceutical compositions may be determined experimentally based on the activities of the carrier(s) and the desired characteristics of the formulation, such as stability and/or minimal oxidation.

In some embodiments, the pharmaceutical composition comprises acetic acid.

In some embodiments, the pharmaceutical composition comprises acetic acid at a concentration of from about 1 mM to about 50 mM.

In some embodiments, the pharmaceutical composition comprises acetic acid at a concentration of from about 10 mM to about 40 mM.

In some embodiments, the pharmaceutical composition comprises acetic acid at a concentration of about 10 mM, about 15 mM, about 20 mM, about 25 mM, about 30 mM, about 35 mM, about 40 mM, about 45 mM or about 50 mM.

In some embodiments, the pharmaceutical composition comprises acetic acid at a concentration of about 25 mM.

In some embodiments, the pharmaceutical composition comprises sodium chloride (NaCl).

In some embodiments, the pharmaceutical composition comprises NaCl at a concentration of from about 20 mM to about 100 mM.

In some embodiments, the pharmaceutical composition comprises NaCl at a concentration of from about 40 mM to about 80 mM.

In some embodiments, the pharmaceutical composition comprises NaCl at a concentration of about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.

In some embodiments, the pharmaceutical composition comprises NaCl at a concentration of about 60 mM.

In some embodiments, the pharmaceutical composition comprises saccharide.

In some embodiments, saccharide is sucrose.

In some embodiments, saccharide is sorbitol.

In some embodiments, saccharide is mannitol.

In some embodiments, the pharmaceutical composition comprises saccharide at a concentration of from about 50 mM to about 500 mM.

In some embodiments, the pharmaceutical composition comprises saccharide at a concentration of from about 50 mM to about 450 mM.

In some embodiments, the pharmaceutical composition comprises saccharide at a concentration of from about 50 mM to about 400 mM.

In some embodiments, the pharmaceutical composition comprises saccharide at a concentration of from about 50 mM to about 350 mM.

In some embodiments, the pharmaceutical composition comprises saccharide at a concentration of from about 100 mM to about 350 mM.

In some embodiments, the pharmaceutical composition comprises saccharide at a concentration of from about 100 mM to about 300 mM.

In some embodiments, the pharmaceutical composition comprises saccharide at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, the pharmaceutical composition comprises mannitol.

In some embodiments, the pharmaceutical composition comprises mannitol at a concentration of from about 100 mM to about 180 mM.

In some embodiments, the pharmaceutical composition comprises mannitol at a concentration of from about 120 mM to about 160 mM.

In some embodiments, the pharmaceutical composition comprises mannitol at a concentration of about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM or about 180 mM.

In some embodiments, the pharmaceutical composition comprises mannitol at a concentration of about 140 mM.

In some embodiments, the pharmaceutical composition comprises polysorbate.

In some embodiments, the pharmaceutical composition comprises polysorbate-20 (PS-20).

In some embodiments, the pharmaceutical composition comprises polysorbate-20 (PS-20) at a concentration of from about 0.01% w/v to about 0.1% w/v.

In some embodiments, the pharmaceutical composition comprises polysorbate-20 (PS-20) at a concentration of from about 0.01% w/v to about 0.08% w/v.

In some embodiments, the pharmaceutical composition comprises polysorbate-20 (PS-20) at a concentration of from about 0.01% w/v to about 0.04% w/v.

In some embodiments, the pharmaceutical composition comprises polysorbate-20 (PS-20) at a concentration of about 0.01% w/v, 0.02% w/v, 0.03% w/v, 0.04% w/v, 0.05% w/v, 0.06% w/v, 0.07% w/v, 0.08% w/v, 0.09% w/v or 0.1% w/v.

The invention also provides a pharmaceutical composition comprising
from about 20 mg/mL to about 120 mg/mL of the anti-CD38 antibody comprising the VH of SEQ ID NO: 4 and the VL of SEQ ID NO: 5 in about 25 mM acetic acid, about 60 mM sodium chloride, about 140 mM mannitol and about 0.04% w/v polysorbate-20 (PS-20); at pH about 5.5; and
about from 30,000 U to about 45,000 U of the hyaluronidase in 10 mM L-Histidine, 130 mM NaCl, 10 mM L-Methionine, 0.02% Polysorbate 80, pH 6.5.

In some embodiments, the hyaluronidase is rHuPH20 (SEQ ID NO: 22).

In some embodiments, the pharmaceutical composition is a non-fixed combination.

The invention also provides a pharmaceutical composition comprising
about 20 mg/mL of the anti-CD38 antibody comprising the VH of SEQ ID NO: 4 and the VL of SEQ ID NO: 5 in about 25 mM acetic acid, about 60 mM sodium chloride, about 140 mM mannitol and about 0.04% w/v polysorbate-20 (PS-20); at pH about 5.5; and about 30,000 U of the hyaluronidase in 10 mM L-Histidine, 130 mM NaCl, 10 mM L-Methionine, 0.02% Polysorbate 80, pH 6.5.

In some embodiments, the hyaluronidase is rHuPH20 (SEQ ID NO: 22).

In some embodiments, the pharmaceutical composition is a non-fixed combination.

The invention also provides a pharmaceutical composition comprising
about 20 mg/mL of the anti-CD38 antibody comprising the VH of SEQ ID NO: 4 and the VL of SEQ ID NO: 5 in about 25 mM acetic acid, about 60 mM sodium chloride, about 140 mM mannitol and about 0.04% w/v polysorbate-20 (PS-20); at pH about 5.5; and
about 45,000 U of the hyaluronidase in 10 mM L-Histidine, 130 mM NaCl, 10 mM L-Methionine, 0.02% Polysorbate 80, pH 6.5.

In some embodiments, the hyaluronidase is rHuPH20 (SEQ ID NO: 22).

In some embodiments, the pharmaceutical composition is a non-fixed combination.

In some embodiments, the pharmaceutical composition comprises histidine.

In some embodiments, the pharmaceutical composition comprises histidine at a concentration of from about 1 mM to about 50 mM.

In some embodiments, the pharmaceutical composition comprises histidine at a concentration of from about 5 mM to about 50 mM.

In some embodiments, the pharmaceutical composition comprises histidine at a concentration of from about 5 mM to about 30 mM.

In some embodiments, the pharmaceutical composition comprises histidine at a concentration of from about 5 mM to about 20 mM.

In some embodiments, the pharmaceutical composition comprises histidine at a concentration of from about 5 mM to about 15 mM.

In some embodiments, the pharmaceutical composition comprises histidine at a concentration of from about 5 mM to about 10 mM.

In some embodiments, the pharmaceutical composition comprises histidine at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM or about 50 mM.

In some embodiments, the pharmaceutical composition comprises histidine at a concentration of about 5 mM.

In some embodiments, the pharmaceutical composition comprises histidine at a concentration of about 10 mM.

In some embodiments, the pharmaceutical composition comprises histidine at a concentration of about 15 mM.

In some embodiments, the pharmaceutical composition comprises histidine at a concentration of about 20 mM.

In some embodiments, the pharmaceutical composition comprises sorbitol at a concentration of from about 50 mM to about 500 mM.

In some embodiments, the pharmaceutical composition comprises sorbitol at a concentration of from about 50 mM to about 450 mM.

In some embodiments, the pharmaceutical composition comprises sorbitol at a concentration of from about 50 mM to about 400 mM.

In some embodiments, the pharmaceutical composition comprises sorbitol at a concentration of from about 50 mM to about 350 mM.

In some embodiments, the pharmaceutical composition comprises sorbitol at a concentration of from about 100 mM to about 350 mM.

In some embodiments, the pharmaceutical composition comprises sorbitol at a concentration of from about 100 mM to about 300 mM.

In some embodiments, the pharmaceutical composition comprises sorbitol at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, the pharmaceutical composition comprises sorbitol at a concentration of about 50 mM.

In some embodiments, the pharmaceutical composition comprises sorbitol at a concentration of about 100 mM.

In some embodiments, the pharmaceutical composition comprises sorbitol at a concentration of about 150 mM.

In some embodiments, the pharmaceutical composition comprises sorbitol at a concentration of about 200 mM.

In some embodiments, the pharmaceutical composition comprises sorbitol at a concentration of about 250 mM.

In some embodiments, the pharmaceutical composition comprises sorbitol at a concentration of about 300 mM.

In some embodiments, the pharmaceutical composition comprises sorbitol at a concentration of about 350 mM.

In some embodiments, the pharmaceutical composition comprises sorbitol at a concentration of about 400 mM.

In some embodiments, the pharmaceutical composition comprises sucrose at a concentration of from about 50 mM to about 500 mM.

In some embodiments, the pharmaceutical composition comprises sucrose at a concentration of from about 50 mM to about 450 mM.

In some embodiments, the pharmaceutical composition comprises sucrose at a concentration of from about 50 mM to about 400 mM.

In some embodiments, the pharmaceutical composition comprises sucrose at a concentration of from about 50 mM to about 350 mM.

In some embodiments, the pharmaceutical composition comprises sucrose at a concentration of from about 100 mM to about 350 mM.

In some embodiments, the pharmaceutical composition comprises sucrose at a concentration of from about 100 mM to about 200 mM.

In some embodiments, the pharmaceutical composition comprises sucrose at a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM or about 500 mM.

In some embodiments, the pharmaceutical composition comprises sucrose at a concentration of about 50 mM.

In some embodiments, the pharmaceutical composition comprises sucrose at a concentration of about 100 mM.

In some embodiments, the pharmaceutical composition comprises sucrose at a concentration of about 150 mM.

In some embodiments, the pharmaceutical composition comprises sucrose at a concentration of about 200 mM.

In some embodiments, the pharmaceutical composition comprises sucrose at a concentration of about 250 mM.

In some embodiments, the pharmaceutical composition comprises sucrose at a concentration of about 300 mM.

In some embodiments, the pharmaceutical composition comprises sucrose at a concentration of about 350 mM.

In some embodiments, the pharmaceutical composition comprises sucrose at a concentration of about 400 mM.

In some embodiments, the pharmaceutical composition comprises methionine.

In some embodiments, the pharmaceutical composition comprises methionine at a concentration of from about 0.1 mg/mL to about 5 mg/mL.

In some embodiments, the pharmaceutical composition comprises methionine at a concentration of from about 0.1 mg/mL to about 2.5 mg/mL.

In some embodiments, the pharmaceutical composition comprises methionine at a concentration of from about 1 mg/mL to about 2 mg/mL.

In some embodiments, the pharmaceutical composition comprises methionine at a concentration of about 0.5 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1/7 mg/mL, about 1.8 mg/mL, about 1.9 mg/mL, about 2.0 mg/mL, about 2.1 mg/mL, about 2.2 mg/mL, about 2/3 mg/mL, about 2.4 mg/mL, about 2.5 mg/mL, about 2.6 mg/mL, about 2.7 mg/mL, about 2.8 mg/mL, about 2.9 mg/mL, about 3 mg/mL, about 3.5 mg/mL, about 4 mg/mL, about 4.5 mg/mL or about 5 mg/mL.

In some embodiments, the pharmaceutical composition is at pH 5.0 to 6.0.

In some embodiments, the pharmaceutical composition is at pH 5.3 to 5.8.

In some embodiments, the pharmaceutical composition is at pH 5.5.

In some embodiments, the pharmaceutical composition is at pH 5.6.

The invention also provides a pharmaceutical composition comprising
from about 1 mg/mL to about 180 mg/mL of the anti-CD38 antibody;
from about 50 U/mL to about 5,000 U/mL of the hyaluronidase
from about 5 mM to about 50 mM histidine; and
from about 50 mM to about 400 mM sorbitol.

In some embodiments, the hyaluronidase is rHuPH20.

The invention also provides a pharmaceutical composition comprising
from about 1 mg/mL to about 180 mg/mL of the anti-CD38 antibody;
from about 50 U/mL to about 5,000 U/mL of the hyaluronidase
from about 5 mM to about 50 mM histidine;
from about 50 mM to about 400 mM sorbitol;
from about 0.01% w/v to about 0.1% PS-20; and
from about 0.1 mg/mL to about 2.5 mg/mL methionine.

In some embodiments, the hyaluronidase is rHuPH20.

The invention also provides a pharmaceutical composition comprising
from about 100 mg/mL to about 120 mg/mL of the anti-CD38 antibody;
from about 50 U/mL to about 5,000 U/mL of the hyaluronidase;
about 10 mM histidine; and
from about 100 mM to about 300 mM sorbitol.

In some embodiments, the hyaluronidase is rHuPH20.

In some embodiments, the pharmaceutical composition further comprises from

about 0.01% w/v to about 0.04% w/v PS-20.

In some embodiments, the pharmaceutical composition further comprises from about 1 mg/mL to about 2 mg/mL methionine.

In some embodiments, the pharmaceutical composition further comprises from about 100 mM to about 200 mM sucrose.

In some embodiments, the anti-CD38 antibody comprises
the HCDR1, the HCDR2, the HCDR3, the LCDR1, the LCDR2 and the LCDR3 of SEQ ID NOs: 6, 7, 8, 9, 10 and 11, respectively;
the VH and the VL of SEQ ID NOs: 4 and 5, respectively; and/or
the heavy chain and the light chain of SEQ ID NOs: 12 and 13, respectively.

In some embodiments, the anti-CD38 antibody comprises
the VH and the VL of SEQ ID NOs: 14 and 15, respectively;
the VH and the VL of SEQ ID NOs: 16 and 17, respectively;
the VH and the VL of SEQ ID NOs: 18 and 19, respectively; or
the VH and the VL of SEQ ID NOs: 20 and 21, respectively;

In some embodiments, the hyaluronidase comprises rHuPH20 (SEQ ID NO: 22)

The invention also provides a pharmaceutical composition comprising
from about 1 mg/mL to about 180 mg/mL of the anti-CD38 antibody comprising the VH and the VL of SEQ ID NOs: 4 and 5, respectively;
from about 50 U/mL to about 5,000 U/mL of the hyaluronidase
from about 5 mM to about 50 mM histidine; and
from about 50 mM to about 400 mM sorbitol.

In some embodiments, the hyaluronidase is rHuPH20.

The invention also provides a pharmaceutical composition comprising
from about 1 mg/mL to about 180 mg/mL of the anti-CD38 antibody comprising the VH and the VL of SEQ ID NOs: 4 and 5, respectively;
from about 50 U/mL to about 5,000 U/mL hyaluronidase
from about 5 mM to about 50 mM histidine;
from about 50 mM to about 400 mM sorbitol;
from about 0.01% w/v to about 0.1% PS-20; and
from about 0.1 mg/mL to about 2.5 mg/mL methionine.

In some embodiments, the hyaluronidase is rHuPH20.

The invention also provides a pharmaceutical composition comprising
from about 100 mg/mL to about 120 mg/mL of the anti-CD38 antibody comprising the VH and the VL of SEQ ID NOs: 4 and 5, respectively;
from about 50 U/mL to about 5,000 U/mL rHuPH20;
about 10 mM histidine;
from about 100 mM to about 300 mM sorbitol;
from about 0.01% w/v to about 0.04% w/v PS-20; and from about 1 mg/mL to about 2 mg/mL methionine.

The invention also provides a pharmaceutical composition comprising
about 100 mg/mL of the anti-CD38 antibody comprising the VH and the VL of SEQ ID NOs: 4 and 5, respectively;
about 500 U/mL rHuPH20;
about 10 mM histidine;
about 300 mM sorbitol;
about 0.04% w/v PS-20; and
about 2 mg/mL methionine; at pH about 5.5.

The invention also provides a pharmaceutical composition comprising
about 120 mg/mL of the anti-CD38 antibody comprising the VH and the VL of SEQ ID NOs: 4 and 5, respectively;
about 2,000 U/mL rHuPH20;
about 10 mM histidine;
about 300 mM sorbitol;
about 0.04% w/v PS-20; and
about 1 mg/mL methionine; at pH about 5.6.

The invention also provides a pharmaceutical composition comprising
about 100 mg/mL of the anti-CD38 antibody comprising the VH and the VL of SEQ ID NOs: 4 and 5, respectively;
about 500 U/mL rHuPH20;
about 10 mM histidine;
about 300 mM sorbitol; and
about 2 mg/mL methionine; at pH about 5.5.

The invention also provides a pharmaceutical composition comprising
about 100 mg/mL of the anti-CD38 antibody comprising the VH and the VL of SEQ ID NOs: 4 and 5, respectively;
about 500 U/mL rHuPH20;
about 10 mM histidine;
about 300 mM sorbitol;
about 0.01% w/v PS-20; and
about 2 mg/mL methionine; at pH about 5.5.

The invention also provides a pharmaceutical composition comprising
about 100 mg/mL of the anti-CD38 antibody comprising the VH and the VL of SEQ ID NOs: 4 and 5, respectively;
about 500 U/mL rHuPH20;
about 10 mM histidine;
about 300 mM sorbitol;
about 0.02% w/v PS-20; and
about 2 mg/mL methionine; at pH about 5.5.

The invention also provides a pharmaceutical composition comprising
about 100 mg/mL of the anti-CD38 antibody comprising the VH and the VL of SEQ ID NOs: 4 and 5, respectively;
about 500 U/mL rHuPH20;
about 10 mM histidine;
about 300 mM sorbitol;
about 0.06% w/v PS-20; and
about 2 mg/mL methionine; at pH about 5.5.

The invention also provides a pharmaceutical composition comprising
about 100 mg/mL of the anti-CD38 antibody comprising the VH and the VL of SEQ ID NOs: 4 and 5, respectively;
about 50 U/mL rHuPH20;
about 10 mM histidine;
about 300 mM sorbitol;
about 0.04% w/v PS-20; and
about 1 mg/mL methionine; at pH about 5.5.

The invention also provides a pharmaceutical composition comprising
about 100 mg/mL of the anti-CD38 antibody comprising the VH and the VL of SEQ ID NOs: 4 and 5, respectively;
about 500 U/mL rHuPH20;
about 10 mM histidine;
about 300 mM sorbitol;
about 0.04% w/v PS-20; and
about 1 mg/mL methionine; at pH about 5.5.

The invention also provides a pharmaceutical composition comprising
about 100 mg/mL of the anti-CD38 antibody comprising the VH and the VL of SEQ ID NOs: 4 and 5, respectively;
about 2,000 U/mL rHuPH20;
about 10 mM histidine;
about 300 mM sorbitol;
about 0.04% w/v PS-20; and
about 1 mg/mL methionine; at pH about 5.5.

The invention also provides a pharmaceutical composition comprising
about 100 mg/mL of the anti-CD38 antibody comprising the VH and the VL of SEQ ID NOs: 4 and 5, respectively;
about 5,000 U/mL rHuPH20;
about 10 mM histidine;
about 300 mM sorbitol;
about 0.04% w/v PS-20; and
about 1 mg/mL methionine; at pH about 5.5.

In some embodiments, the pharmaceutical composition is a fixed combination.

The formulations to be used for in vivo administration are generally sterile. Sterility may be readily accomplished, e.g., by filtration through sterile filtration membranes.

The pharmaceutical compositions of the invention may be prepared by known methods. For example, the pharmaceutical compositions may be prepared, e.g., by dissolving, suspending or emulsifying the anti-CD38 antibody in a sterile aqueous medium or an oily medium conventionally used for injections.

### Administration

The pharmaceutical compositions of the invention may be administered as a non-fixed combination.

The pharmaceutical compositions of the invention may also be administered as a fixed combination, e.g., as a unit dosage form (or dosage unit form). Fixed combinations may be advantageous for ease of administration and uniformity of dosage.

The invention also provides a unit dosage form, comprising the anti-CD38 antibody comprising the VH of SEQ ID NO: 4 and the VL of SEQ ID NO: 5 in an amount of from about 1,200 mg to about 5,000 mg and rHuPH20 in an amount of from about 30,000 U to about 75,000 U.

The invention also provides a unit dosage form, comprising the anti-CD38 antibody comprising the VH of SEQ ID NO: 4 and the VL of SEQ ID NO: 5 in an amount of from about 1,200 mg to about 4,000 mg and rHuPH20 in an amount of from about 30,000 U to about 75,000 U.

The invention also provides a unit dosage form, comprising the anti-CD38 antibody comprising the VH of SEQ ID NO: 4 and the VL of SEQ ID NO: 5 in an amount of from about 1,200 mg to about 2,400 mg and rHuPH20 in an amount of from about 30,000 U to about 45,000 U.

The invention also provides a unit dosage form, comprising the anti-CD38 antibody comprising the VH of SEQ ID NO: 4 and the VL of SEQ ID NO: 5 in an amount of from about 1,200 mg to about 1,800 mg and rHuPH20 in an amount of from about 30,000 U to about 45,000 U.

The invention also provides a unit dosage form, comprising
the anti-CD38 antibody comprising the VH of SEQ ID NO: 4 and the VL of SEQ ID NO: 5 in an amount of from about 1,200 mg to about 5,000 mg;
rHuPH20 in an amount of from about 30,000 U to about 75,000 U;
histidine at a concentration of from about 5 mM to about 15 mM;
sorbitol at a concentration of from about 100 mM to about 300 mM; PS-20 at a concentration of from about 0.01% w/v to about 0.04% w/v; and
methionine at a concentration of from about 1 mg/mL to about 2 mg/mL, at a pH of about 5.5.

The invention also provides a unit dosage form, comprising
the anti-CD38 antibody comprising the VH of SEQ ID NO: 4 and
the VL of SEQ ID NO: 5 in an amount of about 1,200 mg to about 2,400 mg;
rHuPH20 in an amount of from about 30,000 U to about 45,000 U; histidine at a concentration of about 10 mM;
sorbitol at a concentration of about 300 mM;
PS-20 at a concentration of about 0.04% w/v; and
methionine at a concentration of from about 1 mg/mL; at a pH of about 5.5.

The invention also provides a unit dosage form, comprising
the anti-CD38 antibody comprising the VH of SEQ ID NO: 4 and
the VL of SEQ ID NO: 5 in an amount of about 1,200 mg to about 1,800 mg;
rHuPH20 in an amount of from about 30,000 U to about 45,000 U;
histidine at a concentration of about 10 mM;
sorbitol at a concentration of about 300 mM;
PS-20 at a concentration of about 0.04% w/v; and
methionine at a concentration of from about 1 mg/mL; at a pH of about 5.5.

The invention also provides a unit dosage form, comprising
the anti-CD38 antibody comprising the VH of SEQ ID NO: 4 and
the VL of SEQ ID NO: 5 in an amount of from about 1,200 mg to about 1,800 mg;
rHuPH20 in an amount of from about 30,000 U to about 45,000 U;
histidine at a concentration of from about 5 mM to about 15 mM;
sorbitol at a concentration of from about 100 mM to about 300 mM;
PS-20 at a concentration of from about 0.01% w/v to about 0.04% w/v; and
methionine at a concentration of from about 1 mg/mL to about 2 mg/mL, at a pH of about 5.5.

The invention also provides a unit dosage form, comprising
the anti-CD38 antibody comprising the VH of SEQ ID NO: 4 and
the VL of SEQ ID NO: 5 in an amount of about 1,800 mg;
rHuPH20 in an amount of from about 30,000 U;
histidine at a concentration of about 10 mM;
sorbitol at a concentration of about 300 mM;
PS-20 at a concentration of about 0.04% w/v; and
methionine at a concentration of from about 1 mg/mL; at a pH of about 5.5.

The invention also provides a unit dosage form, comprising
the anti-CD38 antibody comprising the VH of SEQ ID NO: 4 and
the VL of SEQ ID NO: 5 in an amount of about 1,800 mg;
rHuPH20 in an amount of from about 45,000 U;
histidine at a concentration of about 10 mM;
sorbitol at a concentration of about 300 mM;
PS-20 at a concentration of about 0.04% w/v; and
methionine at a concentration of from about 1 mg/mL; at a pH of about 5.5.

The pharmaceutical composition of the invention may be administered in a total volume of about 80 mL, 90 mL, 100 mL, 110 mL or 120 mL.

The pharmaceutical composition of the invention may be administered in a total volume of about 10 mL, 11 mL, 12 mL, 13 mL, 14 mL, 15 mL, 16 mL, 17 mL, 18 mL, 19 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 105 mL, 110 mL, 115 mL or 120 mL.

The pharmaceutical composition of the invention may be administered in a total volume of about 10 mL.

The pharmaceutical composition of the invention may be administered in a total volume of about 15 mL.

The pharmaceutical composition of the invention may be administered in a total volume of about 20 mL.

The total volume of administration may be typically smaller for the fixed combinations when compared to the non-fixed combinations.

The invention also provides a container comprising the pharmaceutical composition of the invention.

The invention also provides a container comprising the unit dosage form of the invention.

The container may be a vial, a cartridge, a syringe, a prefilled syringe or a disposable pen.

The administration of the pharmaceutical compositions of the invention may be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose. For example, the pharmaceutical compositions of the invention may be administered once weekly for eight weeks, followed by once in two weeks for 16 weeks, followed by once in four weeks.

The pharmaceutical composition of the invention may be administered subcutaneously.

The pharmaceutical composition of the invention may be administered subcutaneously to the abdominal region.

Subcutaneous administration may be accomplished using a device. The device may be a syringe, a prefilled syringe, an auto-injector, either disposable or reusable, a pen injector, a patch injector, a wearable injector or an ambulatory syringe infusion pump with subcutaneous infusion sets.

For non-fixed combinations, 20 mg/mL anti-CD38 antibody in 25 mM sodium acetate, 60 mM sodium chloride, 140 mM D-mannitol, 0.04% polysorbate 20, pH 5.5 may be mixed with 1 mg/mL (75-150 kU/mL) rHuPH20 in 10 mM L-Histidine, 130 mM NaCl, 10 mM L-Methionine, 0.02% polysorbate-80, pH 6.5 prior to administration of the mixture to a subject.

The pharmaceutical compositions of the invention may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

### Methods of Treatment

The invention also provides a method of treating a cancer, comprising administering to a subject in need thereof the pharmaceutical composition of the invention for a time sufficient to treat the cancer.

In some embodiments, the cancer is a CD38-positive hematological malignancy.

In some embodiments, the CD38-positive hematological malignancy is multiple myeloma.

In some embodiments, the CD38-positive hematological malignancy is diffuse large B-cell lymphoma (DLBCL).

In some embodiments, the CD38-positive hematological malignancy is non-Hodgkin's lymphoma.

In some embodiments, the CD38-positive hematological malignancy is acute lymphoblastic leukemia (ALL).

In some embodiments, the CD38-positive hematological malignancy is follicular lymphoma (FL).

In some embodiments, the CD38-positive hematological malignancy is Burkitt's lymphoma (BL).

In some embodiments, the CD38-positive hematological malignancy is mantle cell lymphoma (MCL).

In some embodiments, the CD38-positive hematological malignancy is light chain amyloidosis (AL).

In some embodiments, the CD38-positive hematological malignancy is multiple myeloma, acute lymphoblastic leukemia (ALL), non-Hodgkin's lymphoma, diffuse large B-cell lymphoma (DLBCL), Burkitt's lymphoma (BL), follicular lymphoma (FL) or mantle-cell lymphoma (MCL).

Examples of B-cell non-Hodgkin's lymphomas are lymphomatoid granulomatosis, primary effusion lymphoma, intravascular large B-cell lymphoma, mediastinal large B-cell lymphoma, heavy chain diseases (including γ, μ, and a disease), lymphomas induced by therapy with immunosuppressive agents, such as cyclosporine-induced lymphoma, and methotrexate-induced lymphoma.

In some embodiments, the cancer is a solid tumor.

The invention also provides a method of treating a CD38-positive hematological malignancy, comprising administering to a subject in need thereof a pharmaceutical composition comprising an anti-CD38 antibody and a hyaluronidase subcutaneously for a time sufficient to treat the CD38-positive hematological malignancy, wherein the anti-CD38 antibody concentration in the pharmaceutical composition is about 20 mg/mL.

The invention also provides a method of treating a CD38-positive hematological malignancy, comprising administering to a subject in need thereof a pharmaceutical composition comprising the anti-CD38 antibody comprising the VH of SEQ ID NO: 4 and the VL of SEQ ID NO: 5 and the hyaluronidase rHuPH20 of SEQ ID NO: 22 subcutaneously for a time sufficient to treat the CD38-positive hematological malignancy, wherein the anti-CD38 antibody concentration in the pharmaceutical composition is about 20 mg/mL.

The invention also provides a method of treating a CD38-positive hematological malignancy, comprising administering to a subject in need thereof a pharmaceutical composition comprising between about 1,200 mg-1,800 mg of the anti-CD38 antibody comprising the VH of SEQ ID NO: 4 and the VL of SEQ ID NO: 5, and between about 30,000 U-45,000 U of the hyaluronidase rHuPH20 of SEQ ID NO: 22 for a time sufficient to treat the CD38-positive hematological malignancy, wherein the anti-CD38 antibody concentration in the pharmaceutical composition is about 20 mg/mL.

The invention also provides a method of treating a CD38-positive hematological malignancy, comprising administering to a subject in need thereof a pharmaceutical composition comprising between about 1,200 mg and about 1,800 mg of the anti-CD38 antibody comprising the VH of SEQ ID NO: 4 and the VL of SEQ ID NO: 5, and about 30,000 U of the hyaluronidase rHuPH20 of SEQ ID NO: 22 for a time sufficient to treat the CD38-positive hematological malignancy, wherein the anti-CD38 antibody concentration in the pharmaceutical composition is about 20 mg/mL.

The invention also provides a method of treating a CD38-positive hematological malignancy, comprising administering to a subject in need thereof a pharmaceutical composition comprising between about 1,200 mg and about 1,800 mg of the anti-CD38 antibody comprising the VH of SEQ ID NO: 4 and the VL of SEQ ID NO: 5, and about 45,000 U of the hyaluronidase rHuPH20 of SEQ ID NO: 22 for a time sufficient to treat the CD38-positive hematological malignancy, wherein the anti-CD38 antibody concentration in the pharmaceutical composition is about 20 mg/mL.

The invention also provides a method of treating a CD38-positive hematological malignancy, comprising administering to a subject in need thereof between about 1,600 mg of the anti-CD38 antibody comprising the VH of SEQ ID NO: 4 and the VL of SEQ ID NO: 5, and about 30,000 U of the hyaluronidase rHuPH20 of SEQ ID NO: 22 for a time sufficient to treat the CD38-positive hematological malignancy, wherein the anti-CD38 antibody concentration in the pharmaceutical composition is about 20 mg/mL.

The invention also provides a method of treating a CD38-positive hematological malignancy, comprising administering to a subject in need thereof about 1,600 mg of the anti-CD38 antibody comprising the VH of SEQ ID NO: 4 and the VL of SEQ ID NO: 5, and about 45,000 U of the hyaluronidase rHuPH20 of SEQ ID NO: 22 for a time sufficient to treat the CD38-positive hematological malignancy, wherein the anti-CD38 antibody concentration in the pharmaceutical composition is about 20 mg/mL.

The invention also provides a method of treating a CD38-positive hematological malignancy, comprising administering to a subject in need thereof a pharmaceutical composition comprising the anti-CD38 antibody comprising the VH of SEQ ID NO: 4 and the VL of SEQ ID NO: 5 and the hyaluronidase, wherein the pharmaceutical composition is a non-fixed combination.

The invention also provides a method of treating a CD38-positive hematological malignancy, comprising administering to a subject in need thereof a pharmaceutical composition comprising
from about 20 mg/mL to about 120 mg/mL of the anti-CD38 antibody comprising the VH of SEQ ID NO: 4 and the VL of SEQ ID NO: 5 in about 25 mM acetic acid, about 60 mM sodium chloride, about 140 mannitol and about 0.04% w/v polysorbate-20 (PS-20); at pH about 5.5; and
about from 30,000 U to about 45,000 U of the hyaluronidase in 10 mM L-histidine, 130 mM NaCl, 10 mM L-methionine, 0.02% Polysorbate-80, pH 6.5.

In some embodiments, the hyaluronidase is rHuPH20.

In some embodiments, the pharmaceutical composition is a non-fixed combination.

The invention also provides a method of treating a CD38-positive hematological malignancy, comprising administering to a subject in need thereof a pharmaceutical composition comprising
about 20 mg/mL of the anti-CD38 antibody comprising the VH of SEQ ID NO: 4 and the VL of SEQ ID NO: 5 in about 25 mM acetic acid, about 60 mM sodium chloride, about 140 mannitol and about 0.04% w/v polysorbate-20 (PS-20); at pH about 5.5; and
about 30,000 U of the hyaluronidase in 10 mM L-histidine, 130 mM NaCl, 10 mM L-methionine, 0.02% Polysorbate-80, pH 6.5.

In some embodiments, the hyaluronidase is rHuPH20.

In some embodiments, the pharmaceutical composition is a non-fixed combination.

The invention also provides a method of treating a CD38-positive hematological malignancy, comprising administering to a subject in need thereof a pharmaceutical composition comprising
about 20 mg/mL of the anti-CD38 antibody comprising the VH of SEQ ID NO: 4 and the VL of SEQ ID NO: 5 in about 25 mM acetic acid, about 60 mM sodium chloride, about 140 mannitol and about 0.04% w/v polysorbate-20 (PS-20); at pH about 5.5; and
about 45,000 U of the hyaluronidase in 10 mM L-histidine, 130 mM NaCl, 10 mM L-methionine, 0.02% Polysorbate-80, pH 6.5.

In some embodiments, the hyaluronidase is rHuPH20.

In some embodiments, the pharmaceutical composition is a non-fixed combination.

The invention also provides a method of treating a CD38-positive hematological malignancy, comprising administering to a subject in need thereof a pharmaceutical composition comprising the anti-CD38 antibody comprising the VH of SEQ ID NO: 4 and the VL of SEQ ID NO: 5 and the hyaluronidase, wherein the pharmaceutical composition is a fixed combination.

The invention also provides a method of treating a CD38-positive hematological malignancy, comprising administering to a subject in need thereof a pharmaceutical composition comprising
from about 1 mg/mL to about 180 mg/mL of the anti-CD38 antibody comprising the VH and the VL of SEQ ID NOs: 4 and 5, respectively;
from about 50 U/mL to about 5,000 U/mL of the hyaluronidase
from about 5 mM to about 50 mM histidine; and
from about 50 mM to about 400 mM sorbitol.

In some embodiments, the hyaluronidase is rHuPH20.

The invention also provides a method of treating a CD38-positive hematological malignancy, comprising administering to a subject in need thereof a pharmaceutical composition comprising
from about 1 mg/mL to about 180 mg/mL of the anti-CD38 antibody comprising the VH and the VL of SEQ ID NOs: 4 and 5, respectively;
from about 50 U/mL to about 5,000 U/mL of the hyaluronidase
from about 5 mM to about 50 mM histidine;
from about 50 mM to about 400 mM sorbitol;
from about 0.01% w/v to about 0.1% PS-20; and
from about 0.1 mg/mL to about 2.5 mg/mL methionine.

In some embodiments, the hyaluronidase is rHuPH20.

The invention also provides a method of treating a CD38-positive hematological malignancy, comprising administering to a subject in need thereof a pharmaceutical composition comprising
from about 100 mg/mL to about 120 mg/mL of the anti-CD38 antibody comprising the VH and the VL of SEQ ID NOs: 4 and 5, respectively;
from about 50 U/mL to about 5,000 U/mL of the hyaluronidase;
about 10 mM histidine;
from about 100 mM to about 300 mM sorbitol;
from about 0.01% w/v to about 0.04% w/v PS-20; and
from about 1 mg/mL to about 2 mg/mL methionine.

In some embodiments, the hyaluronidase is rHuPH20.

The invention also provides a method of treating a CD38-positive hematological malignancy, comprising administering to a subject in need thereof a pharmaceutical composition comprising
about 100 mg/mL of the anti-CD38 antibody comprising the VH and the VL of SEQ ID NOs: 4 and 5, respectively;
about 500 U/mL of the hyaluronidase;
about 10 mM histidine;
about 300 mM sorbitol;
about 0.04% w/v PS-20; and
about 2 mg/mL methionine at pH about 5.5.

In some embodiments, the hyaluronidase is rHuPH20.

The invention also provides a method of treating a CD38-positive hematological malignancy, comprising administering to a subject in need thereof a pharmaceutical composition comprising
about 120 mg/mL of the anti-CD38 antibody comprising the VH and the VL of SEQ ID NOs: 4 and 5, respectively;
about 2,000 U/mL rHuPH20;
about 10 mM histidine;
about 300 mM sorbitol;
about 0.04% w/v PS-20; and
about 1 mg/mL methionine; at pH about 5.6.

In some embodiments, the hyaluronidase is rHuPH20.

The anti-CD38 antibodies in the pharmaceutical compositions of the invention may induce killing of CD38-expressing tumor cells by antibody-dependent cell-mediated cytotoxicity (ADCC), antibody-dependent cellular phagocytosis (ADCP), complement dependent cytotoxicity (CDC), apoptosis, or modulation of CD38 enzymatic activity. The anti-CD38 antibodies in the pharmaceutical compositions of the invention may also mediate anti-tumor efficacy by their immunomodulatory effects by inducing CD4⁺ and CD8⁺ T cell proliferation, and/or by relieving inhibition of inflammatory responses mediated by myeloid-derived suppressor cells (MDSCs) and regulatory T cells (Tregs).

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells, monocytes, macrophages and neutrophils via Fc gamma receptors (FcγR) expressed on effector cells. For example, NK cells express FcγRIIIa, whereas monocytes express FcγRI, FcγRII and FcvRIIIa. Death of the antibody-coated target cell, such as CD38-expressing cells, occurs as a result of effector cell activity through the secretion of membrane pore-forming proteins and proteases. To assess ADCC activity of an antibody that specifically binds CD38, the antibody may be added to CD38-expressing cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Exemplary effector cells for such assays include peripheral blood mononuclear cells (PBMC) and NK cells. Exemplary target cells include Tregs or MDSCs expressing CD38. In an exemplary assay, target cells are labeled with 20 μCi of ⁵¹Cr for 2 hours and washed extensively. Cell concentration of the target cells may be adjusted to 1×10⁶ cells/ml, and anti-CD38 antibodies at various concentrations are added. Assays are started by adding target cells at an effector:target cell ratio of 40:1. After incubation for 3 hr at 37° C. assays are stopped by centrifugation, and ⁵¹Cr release from lysed cells are measured in a scintillation counter. Percentage of cellular cytotoxicity may be calculated as % maximal lysis which may be induced by adding 3% perchloric acid to target cells.

“Antibody-dependent cellular phagocytosis” (“ADCP”) refers to a mechanism of elimination of antibody-coated target cells by internalization by phagocytic cells, such as macrophages or dendritic cells. ADCP may be evaluated by using Tregs or MDSCs expressing CD38 as target cells engineered to express GFP or other labeled molecule. Effector:target cell ratio may be for example 4:1. Effector cells may be incubated with target cells for 4 hours with or without anti-CD38 antibody. After incubation, cells may be detached using accutase. Macrophages may be identified with anti-CD11b and anti-CD14 antibodies coupled to a fluorescent label, and percent phagocytosis may be determined based on % GFP fluorescent in the CD11⁺CD14⁺ macrophages using standard methods.

“Complement-dependent cytotoxicity”, or “CDC”, refers to a mechanism for inducing cell death in which an Fc effector domain of a target-bound antibody binds and activates complement component C1q which in turn activates the complement cascade leading to target cell death. Activation of complement may also result in deposition of complement components on the target cell surface that facilitate ADCC by binding complement receptors (e.g., CR3) on leukocytes.

The ability of monoclonal antibodies to induce ADCC may be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%.

The removal of the core fucose from the biantennary complex-type oligosaccharides attached to the Fc regions enhances the ADCC of antibodies via improved FcγRIIIa binding without altering antigen binding or CDC activity. Such mAbs may be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the α 1,6-fucosyltrasferase (FUT8) gene (Mori et al., Biotechnol Bioeng 88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008). ADCC elicited by anti-CD38 antibodies used in the methods of the invention, and in some embodiments of each and every one of the numbered embodiments listed below, may also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

In some embodiments, the anti-CD38 antibody comprises a substitution in the antibody Fc.

In some embodiments, the anti-CD38 antibody comprises a substitution in the antibody Fc at amino acid positions 256, 290, 298, 312, 356, 330, 333, 334, 360, 378 or 430 (residue numbering according to the EU index).

In some embodiments, the anti-CD38 antibody has a biantennary glycan structure with fucose content of about between 0% to about 15%, for example 15%, 14%, 13%, 12%, 11% 10%, 9%, 8%, 7%, 6%, 5%, 4%, 3%, 2%, 1% or 0%.

In some embodiments, the anti-CD38 antibody has a biantennary glycan structure with fucose content of about 50%, 40%, 45%, 40%, 35%, 30%, 25%, 20%, 15%, 14%, 13%, 12%, 11% 10%, 9%, 8%, 7%, 6%, 5%, 4%, 3%, 2%, 1% or 0%

Substitutions in the Fc and reduced fucose content may enhance the ADCC activity of the antibody that specifically binds CD38.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Intl. Pat. Publ. No. WO2008/077546; 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS) or 5) separation of the mAb oligosaccharides from the mAb protein by specific enzymatic deglycosylation with PNGase F at Asn 297. The oligosaccharides released may be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosacharride forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used herein refers to antibodies with fucose content of about 0%-15%.

“Normal fucose” or ‘normal fucose content” as used herein refers to antibodies with fucose content of about over 50%, typically about over 60%, 70%, 80% or over 85%.

In the methods described herein, and in some embodiments of each and every one of the numbered embodiments listed below, the anti-CD38 antibody is of IgG1, IgG2, IgG3 or IgG4 isotype.

Antibodies that are substantially identical to the antibody comprising the VH of SEQ ID NO: 4 and the VL of SEQ ID NO: 5 may be used in the methods of the invention. The term “substantially identical” as used herein means that the two antibody VH or VL amino acid sequences being compared are identical or have “insubstantial differences”. Insubstantial differences are substitutions of 1, 2, 3, 4, 5, 6, 7, 8, 9, 10, 11, 12, 13, 14, or 15 amino acids in an antibody heavy chain or light chain that do not adversely affect antibody properties. Percent identity may be determined for example by pairwise alignment using the default settings of the AlignX module of Vector NTI v.9.0.0 (Invitrogen, Carlsbad, Calif.). The protein sequences of the present invention may be used as a query sequence to perform a search against public or patent databases to, for example, identify related sequences. Exemplary programs used to perform such searches are the XBLAST or BLASTP programs (http_//www_ncbi_nlm/nih_gov), or the GenomeQuest™ (GenomeQuest, Westborough, Mass.) suite using the default settings. Exemplary substitutions that may be made to the anti-CD38 antibodies used in the methods of the invention are for example conservative substitutions with an amino acid having similar charge, hydrophobic, or stereochemical characteristics. Conservative substitutions may also be made to improve antibody properties, for example stability or affinity, or to improve antibody effector functions. 1, 2, 3, 4, 5, 6, 7, 8, 9, 10, 11, 12, 13, 14, or 15 amino acid substitutions may be made for example to the heavy or the light chain of the anti-CD38 antibody. Furthermore, any native residue in the heavy or light chain may also be substituted with alanine, as has been previously described for alanine scanning mutagenesis (MacLennan et al., Acta Physiol Scand Suppl 643:55-67, 1998; Sasaki et al., Adv Biophys 35:1-24, 1998). Desired amino acid substitutions may be determined by those skilled in the art at the time such substitutions are desired. Amino acid substitutions may be done for example by PCR mutagenesis (U.S. Pat. No. 4,683,195). Libraries of variants may be generated using well known methods, for example using random (NNK) or non-random codons, for example DVK codons, which encode 11 amino acids (Ala, Cys, Asp, Glu, Gly, Lys, Asn, Arg, Ser, Tyr, Trp) and screening the libraries for variants with desired properties. The generated variants may be tested for their binding to CD38, their ability to induce ADCC, ADCP or apoptosis, or modulate CD38 enzymatic activity in vitro using methods described herein.

In some embodiments, the anti-CD38 antibody may bind human CD38 with a range of affinities (K_{D}). In one embodiment according to the invention, and in some embodiments of each and every one of the numbered embodiments listed below, the anti-CD38 antibody binds to CD38 with high affinity, for example, with a K_{D} equal to or less than about 10⁻⁷ M, such as but not limited to, 1-9.9 (or any range or value therein, such as 1, 2, 3, 4, 5, 6, 7, 8, or 9)×10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M or any range or value therein, as determined by surface plasmon resonance or the Kinexa method, as practiced by those of skill in the art. One exemplary affinity is equal to or less than 1×10⁻⁸ M. Another exemplary affinity is equal to or less than 1×10⁻⁹ M.

In some embodiments, the anti-CD38 antibody is a bispecific antibody. The VL and/or the VH regions of the existing anti-CD38 antibodies or the VL and VH regions identified de novo as described herein may be engineered into bispecific full length antibodies. Such bispecific antibodies may be made by modulating the CH3 interactions between the monospecific antibody heavy chains to form bispecific antibodies using technologies such as those described in U.S. Pat. No. 7,695,936; Intl. Pat. Publ. No. WO04/111233; U.S. Pat. Publ. No. US2010/0015133; U.S. Pat. Publ. No. US2007/0287170; Intl. Pat. Publ. No. WO2008/119353; U.S. Pat. Publ. No. US2009/0182127; U.S. Pat. Publ. No. US2010/0286374; U.S. Pat. Publ. No. US2011/0123532; Intl. Pat. Publ. No. WO2011/131746; Int. Pat. Publ. No. WO2011/143545; or U.S. Pat. Publ. No. US2012/0149876. Additional bispecific structures into which the VL and/or the VH regions of the antibodies of the invention may be incorporated are for example Dual Variable Domain Immunoglobulins (Inlt. Pat. Publ. No. WO2009/134776), or structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441).

For example, bispecific antibodies may be generated in vitro in a cell-free environment by introducing asymmetrical mutations in the CH3 regions of two monospecific homodimeric antibodies and forming the bispecific heterodimeric antibody from two parental monospecific homodimeric antibodies in reducing conditions to allow disulfide bond isomerization according to methods described in Intl. Pat. Publ. No. WO2011/131746. In the methods, the first monospecific bivalent antibody (e.g., anti-CD38 antibody) and the second monospecific bivalent antibody are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Exemplary CH3 mutations that may be used in a first heavy chain and in a second heavy chain of the bispecific antibody are K409R and/or F405L.

The methods of the invention may be used to treat an animal patient belonging to any classification. Examples of such animals include mammals such as humans, rodents, dogs, cats and farm animals.

### Combination Therapies

The pharmaceutical compositions of the invention may be administered in combination with a second therapeutic agent, or combinations thereof.

The second therapeutic agent may be melphalan, mechlorethamine, thioepa, chlorambucil, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin, thalidomide or a thalidomide analog, lenalidomide or CC4047, a proteasome inhibitor, such as bortezomib or vinca alkaloid, such as vincristine or an anthracycline, such as doxorubicin.

In some embodiments, the second therapeutic agent is a proteasome inhibitor.

In some embodiments, the proteasome inhibitor is bortezomib, carfilzomib or ixazomib.

In some embodiments, the second therapeutic agent is an alkylating agent. In some embodiments, the alkylating agent is busulfan, cyclophosphamide, bendamustine, chlorambucli, carboplatin, cisplatin, temozolomide, melphalan, busulfan, bendamustine, carmustine, lomustine, dacarbazine, oxaliplatin, ifosfamide, mechlorethamine, thiotepa, trabectedin or streptozocin.

In some embodiments, the second therapeutic agent is a glutamic acid derivative.

In some embodiments, the glutamic acid derivative is Revlimid® (lenalidomide), thalidomide or Pomalyst® (pomalidomide).

In some embodiments, the subject is further administered a corticosteroid.

In some embodiments, the corticosteroid is dexamethasone or predisone.

The second therapeutic agent or combinations thereof are typically administered at dosages recommended for the agent.

The pharmaceutical composition of the invention may be administered simultaneously or sequentially with the second thereapeutic agent or combinations thereof.

While having described the invention in general terms, the embodiments of the invention will be further disclosed in the following examples that should not be construed as limiting the scope of the claims.

### EXAMPLE 1

### 1 Indications and Usage

DARZALEX FASPRO is indicated for the treatment of adult patients with multiple myeloma:
in combination with bortezomib, melphalan and prednisone in newly diagnosed patients who are ineligible for autologous stem cell transplant.
in combination with lenalidomide and dexamethasone in newly diagnosed patients who are ineligible for autologous stem cell transplant and in patients with relapsed or refractory multiple myeloma who have received at least one prior therapy.
in combination with bortezomib and dexamethasone in patients who have received at least one prior therapy.
as monotherapy, in patients who have received at least three prior lines of therapy including a proteasome inhibitor (PI) and an immunomodulatory agent or who are double-refractory to a PI and an immunomodulatory agent.

### 2 Dosage and Administration

### 2.1 Important Dosing Information

DARZALEX FASPRO is for subcutaneous use only.
Administer medications before and after administration of DARZALEX FASPRO to minimize administration-related reactions [see Dosage and Administration (2.3)].
Type and screen patients prior to starting DARZALEX FASPRO.

### 2.2 Recommended Dosage

The recommended dose of DARZALEX FASPRO is 1,800 mg/30,000 units (1,800 mg daratumumab and 30,000 units hyaluronidase) administered subcutaneously over approximately 3-5 minutes. Tables 1, 2, and 3 provide the recommended dosing schedule when DARZALEX FASPRO is administered as monotherapy or as part of a combination therapy.
Monotherapy and in Combination with Lenalidomide and Dexamethasone (D-Rd)
Use the dosing schedule provided in Table 1 when DARZALEX FASPRO is administered:
in combination with lenalidomide and dexamethasone (4-week cycle) OR
as monotherapy.

TABLE 1
DARZALEX FASPRO dosing schedule
in combination with lenalidomide and
dexamethasone (4-week cycle) and for monotherapy

| Weeks | Schedule |
|---|---|
| Weeks 1 to 8 | weekly (total of 8 doses) |
| Weeks 9 to 24^{a} | every two weeks (total of 8 doses) |
| Week 25 onwards until | every four weeks |
| disease progression^{b} | |

^{a}First dose of the every-2-week dosing schedule is given at Week 9
^{b}First dose of the every-4-week dosing schedule is given at Week 25

When DARZALEX FASPRO is administered as part of a combination therapy, see Clinical Studies (14.2) and the prescribing information for dosage recommendations for the other drugs.
In Combination with Bortezomib, Melphalan and Prednisone (D-VMP)
Use the dosing schedule provided in Table 2 when DARZALEX FASPRO is administered in combination with bortezomib, melphalan and prednisone (6-week cycle).

TABLE 2
DARZALEX FASPRO dosing schedule
in combination with bortezomib,
melphalan and prednisone (6-week cycle)

| Weeks | Schedule |
|---|---|
| Weeks 1 to 6 | weekly (total of 6 doses) |
| Weeks 7 to 54^{a} | every three weeks (total of 16 doses) |
| Week 55 onwards until | |
| disease progression^{b} | every four weeks |

^{a}First dose of the every-3-week dosing schedule is given at Week 7
^{b}First dose of the every-4-week dosing schedule is given at Week 55

When DARZALEX FASPRO is administered as part of a combination therapy, see Clinical Studies (14.1) and the prescribing information for dosage recommendations for the other drugs.
In Combination with Bortezomib and Dexamethasone (D-Vd)
Use the dosing schedule in Table 3 when DARZALEX FASPRO is administered in combination with bortezomib and dexamethasone (3-week cycle).

TABLE 3
DARZALEX FASPRO dosing schedule
in combination with bortezomib and
dexamethasone (3-week cycle)

| Weeks | Schedule |
|---|---|
| Weeks 1 to 9 | weekly (total of 9 doses) |
| Weeks 10 to 24^{a} | every three weeks (total of 5 doses) |
| Week 25 onwards until | |
| disease progression^{b} | every four weeks |

^{a}First dose of the every-3-week dosing schedule is given at Week 10
^{b}First dose of the every-4-week dosing schedule is given at Week 25

When DARZALEX FASPRO is administered as part of a combination therapy, see the prescribing information for dosage recommendations for the other drugs.

### Missed DARZALEX FASPRO Doses

If a dose of DARZALEX FASPRO is missed, administer the dose as soon as possible and adjust the dosing schedule to maintain the dosing interval.

### 2.3 Recommended Concomitant Medications

### Pre-Medication

Administer the following pre-medications 1-3 hours before each dose of DARZALEX FASPRO:
Acetaminophen 650 to 1,000 mg orally
Diphenhydramine 25 to 50 mg (or equivalent) orally or intravenously
Corticosteroid (long- or intermediate-acting)

Monotherapy
Administer methylprednisolone 100 mg (or equivalent) orally or intravenously. Consider reducing the dose of methylprednisolone to 60 mg (or equivalent) following the second dose of DARZALEX FASPRO.

In Combination
Administer dexamethasone 20 mg (or equivalent) orally or intravenously prior to every DARZALEX FASPRO administration.
When dexamethasone is the background regimen-specific corticosteroid, the dexamethasone dose that is part of the background regimen will serve as pre-medication on DARZALEX FASPRO administration days [see Clinical Studies (14)].
Do not administer background regimen-specific corticosteroids (e.g. prednisone) on DARZALEX FASPRO administration days when patients have received dexamethasone (or equivalent) as a pre-medication.

### Post-Medication

Administer the following post-medications:

Monotherapy
Administer methylprednisolone 20 mg (or an equivalent dose of an intermediate- or long-acting corticosteroid) orally for 2 days starting the day after the administration of DARZALEX FASPRO.

In Combination
Consider administering oral methylprednisolone at a dose of less than or equal to 20 mg (or an equivalent dose of an intermediate- or long-acting corticosteroid) beginning the day after administration of DARZALEX FASPRO.
If a background regimen-specific corticosteroid (e.g. dexamethasone, prednisone) is administered the day after the administration of DARZALEX FASPRO, additional corticosteroids may not be needed [see Clinical Studies (14)].
   If the patient does not experience a major systemic administration-related reaction after the first 3 doses of DARZALEX FASPRO, consider discontinuing the administration of corticosteroids (excluding any background regimen-specific corticosteroid).
   For patients with a history of chronic obstructive pulmonary disease, consider prescribing short and long-acting bronchodilators and inhaled corticosteroids. Following the first 4 doses of DARZALEX FASPRO, consider discontinuing these additional post-medications, if the patient does not experience a major systemic administration-related reaction.

### Prophylaxis for Herpes Zoster Reactivation

Initiate antiviral prophylaxis to prevent herpes zoster reactivation within 1 week after starting DARZALEX FASPRO and continue for 3 months following the end of treatment [see Adverse Reactions (6.1)].

### 2.4 Dosage Modifications for Adverse Reactions

No dose reductions of DARZALEX FASPRO are recommended. Consider withholding DARZALEX FASPRO to allow recovery of blood cell counts in the event of myelosuppression [see Warnings and Precautions (5.2, 5.3)].

### 2.5 Preparation and Administration

DARZALEX FASPRO should be administered by a healthcare provider.
To prevent medication errors, check the vial labels to ensure that the drug being prepared and administered is DARZALEX FASPRO for subcutaneous use. Do not administer DARZALEX FASPRO intravenously.
DARZALEX FASPRO is ready to use.

### Preparation

Remove the DARZALEX FASPRO vial from refrigerated storage [2° C. to 8° C. (36° F. to 46° F.)] and equilibrate to ambient temperature [15° C. to 30° C. (59° F. to 86° F.)]. Store the unpunctured vial at ambient temperature and ambient light for a maximum of 24 hours. Keep out of direct sunlight. Do not shake.
Withdraw 15 mL from the vial into a syringe.
DARZALEX FASPRO is compatible with polypropylene or polyethylene syringe material; polypropylene, polyethylene, or polyvinyl chloride (PVC) subcutaneous infusion sets; and stainless steel transfer and injection needles. Use the product immediately.
After the solution of DARZALEX FASPRO is withdrawn into the syringe, replace the transfer needle with a syringe closing cap. Label the syringe appropriately to include the route of administration per institutional standards. Label the syringe with the peel-off label.
To avoid needle clogging, attach the hypodermic injection needle or subcutaneous infusion set to the syringe immediately prior to injection.
Parenteral drug products should be inspected visually for particulate matter and discoloration prior to administration, whenever solution and container permit. Do not use if opaque particles, discoloration or other foreign particles are present.

### Storage

If the syringe containing DARZALEX FASPRO is not used immediately, store the DARZALEX FASPRO solution for up to 4 hours at ambient temperature and ambient light. Discard after 4 hours, if not used.

### Administration

Inject 15 mL DARZALEX FASPRO into the subcutaneous tissue of the abdomen approximately 3 inches [7.5 cm] to the right or left of the navel over approximately 3-5 minutes. No data are available on performing the injection at other sites of the body.
Rotate injection sites for successive injections.
Never inject DARZALEX FASPRO into areas where the skin is red, bruised, tender, hard or areas where there are scars.
Pause or slow down delivery rate if the patient experiences pain. In the event pain is not alleviated by pausing or slowing down delivery rate, a second injection site may be chosen on the opposite side of the abdomen to deliver the remainder of the dose.
During treatment with DARZALEX FASPRO, do not administer other medications for subcutaneous use at the same site as DARZALEX FASPRO.

### 3 Dosage Forms and Strengths

Injection: 1,800 mg daratumumab and 30,000 units hyaluronidase per 15 mL (120 mg and 2,000 units/mL) colorless to yellow and clear to opalescent solution in a single-dose vial.

### 4 Contraindications

DARZALEX FASPRO is contraindicated in patients with a history of severe hypersensitivity to daratumumab, hyaluronidase or any of the components of the formulation [see Warnings and Precautions (5.1) and Adverse Reactions (6.3)].

### 5 Warnings and Precautions

### 5.1 Hypersensitivity and Other Administration Reactions

Both systemic administration-related reactions, including severe or life-threatening reactions, and local injection-site reactions can occur with DARZALEX FASPRO.

### Systemic Reactions

In a pooled safety population of 490 patients who received DARZALEX FASPRO as monotherapy or in combination, 11% of patients experienced a systemic administration-related reaction (Grade 2: 3.9%, Grade 3: 1.4%). Systemic administration-related reactions occurred in 10% of patients with the first injection, 0.2% with the second injection, and cumulatively 0.8% with subsequent injections. The median time to onset was 3.7 hours (range: 9 minutes to 3.5 days). Of the 84 systemic administration-related reactions that occurred in 52 patients, 73 (87%) occurred on the day of DARZALEX FASPRO administration. Delayed systemic administration-related reactions have occurred in less than 1% of the patients.
Severe reactions included hypoxia, dyspnea, hypertension and tachycardia. Other signs and symptoms of systemic administration-related reactions may include respiratory symptoms, such as bronchospasm, nasal congestion, cough, throat irritation, allergic rhinitis, and wheezing, as well as anaphylactic reaction, pyrexia, chest pain, pruritis, chills, vomiting, nausea, and hypotension.
Pre-medicate patients with histamine-1 receptor antagonist, acetaminophen and corticosteroids [see Dosage and Administration (2.3)]. Monitor patients for systemic administration-related reactions, especially following the first and second injections. For anaphylactic reaction or life-threatening (Grade 4) administration-related reactions, immediately and permanently discontinue DARZALEX FASPRO. Consider administering corticosteroids and other medications after the administration of DARZALEX FASPRO depending on dosing regimen and medical history to minimize the risk of delayed (defined as occurring the day after administration) systemic administration-related reactions [see Dosage and Administration (2.3)].

### Local Reactions

In this pooled safety population, injection-site reactions occurred in 8% of patients, including Grade 2 reactions in 0.6%. The most frequent (>1%) injection-site reaction was injection site erythema. These local reactions occurred a median of 7 minutes (range: 0 minutes to 4.7 days) after starting administration of DARZALEX FASPRO. Monitor for local reactions and consider symptomatic management.

### 5.2 Neutropenia

Daratumumab may increase neutropenia induced by background therapy [see Adverse Reactions (6.1)].
Monitor complete blood cell counts periodically during treatment according to manufacturer's prescribing information for background therapies. Monitor patients with neutropenia for signs of infection. Consider withholding DARZALEX FASPRO until recovery of neutrophils. In lower body weight patients receiving DARZALEX FASPRO, higher rates of Grade 3-4 neutropenia were observed.

### 5.3 Thrombocytopenia

Daratumumab may increase thrombocytopenia induced by background therapy [see Adverse Reactions (6.1)].
Monitor complete blood cell counts periodically during treatment according to manufacturer's prescribing information for background therapies. Consider withholding DARZALEX FASPRO until recovery of platelets.

### 5.4 Embryo-Fetal Toxicity

Based on the mechanism of action, DARZALEX FASPRO can cause fetal harm when administered to a pregnant woman. DARZALEX FASPRO may cause depletion of fetal immune cells and decreased bone density. Advise pregnant women of the potential risk to a fetus. Advise females with reproductive potential to use effective contraception during treatment with DARZALEX FASPRO and for 3 months after the last dose [see Use in Specific Populations (8.1, 8.3)].
The combination of DARZALEX FASPRO with lenalidomide is contraindicated in pregnant women, because lenalidomide may cause birth defects and death of the unborn child. Refer to the lenalidomide prescribing information on use during pregnancy.
5.5 Interference with Serological Testing
Daratumumab binds to CD38 on red blood cells (RBCs) and results in a positive Indirect Antiglobulin Test (Indirect Coombs test). Daratumumab-mediated positive indirect antiglobulin test may persist for up to 6 months after the last daratumumab administration. Daratumumab bound to RBCs masks detection of antibodies to minor antigens in the patient's serum [see References (15)]. The determination of a patient's ABO and Rh blood type are not impacted [see Drug Interactions (7.1)].
Notify blood transfusion centers of this interference with serological testing and inform blood banks that a patient has received DARZALEX FASPRO. Type and screen patients prior to starting DARZALEX FASPRO [see Dosage and Administration (2.1)].
5.6 Interference with Determination of Complete Response
Daratumumab is a human IgG kappa monoclonal antibody that can be detected on both the serum protein electrophoresis (SPE) and immunofixation (IFE) assays used for the clinical monitoring of endogenous M-protein [see Drug Interactions (7.1)]. This interference can impact the determination of complete response and of disease progression in some DARZALEX FASPRO-treated patients with IgG kappa myeloma protein.

### 6 Adverse Reactions

The following clinically significant adverse reactions are described elsewhere in the labeling:
Hypersensitivity and Other Administration Reactions [see Warning and Precautions (5.1)].
Neutropenia [see Warning and Precautions (5.2)].
Thrombocytopenia [see Warning and Precautions (5.3)].

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

### Newly Diagnosed Multiple Myeloma

In Combination with Bortezomib, Melphalan and Prednisone
The safety of DARZALEX FASPRO with bortezomib, melphalan and prednisone (D-VMP) was evaluated in a single-arm cohort of PLEIADES [see Clinical Studies (14.1)]. Patients received DARZALEX FASPRO 1,800 mg/30,000 units administered subcutaneously once weekly from weeks 1 to 6, once every 3 weeks from weeks 7 to 54 and once every 4 weeks starting with week 55 until disease progression or unacceptable toxicity (N=67) in combination with bortezomib, melphalan and prednisone. Among these patients, 93% were exposed for 6 months or longer and 19% were exposed for greater than one year.
Serious adverse reactions occurred in 39% of patients who received DARZALEX FASPRO. Serious adverse reactions in >5% of patients included pneumonia and pyrexia. Fatal adverse reactions occurred in 3.0% of patients.
Permanent discontinuation of DARZALEX FASPRO due to an adverse reaction occurred in 4.5% of patients. The adverse reaction resulting in permanent discontinuation of DARZALEX FASPRO in more than 1 patient was neutropenic sepsis.
Dosage interruptions (defined as dose delays or skipped doses) due to an adverse reaction occurred in 51% of patients who received DARZALEX FASPRO. Adverse reactions requiring dosage interruptions in >5% of patients included thrombocytopenia, neutropenia, anemia, and pneumonia.
The most common adverse reactions (≥20%) were upper respiratory tract infection, constipation, nausea, fatigue, pyrexia, peripheral sensory neuropathy, diarrhea, cough, insomnia, vomiting, and back pain.
Table 4 summarizes the adverse reactions in patients who received DARZALEX FASPRO with bortezomib, melphalan and prednisone (D-VMP) in PLEIADES.

TABLE 4
Adverse Reactions (≥10%) in Patients
Who Received DARZALEX FASPRO with
Bortezomib, Melphalan and Prednisone (D-VMP) in PLEIADES

| | DARZALEX FASPRO | |
|---|---|---|
| | with Bortezomib, Melphalan and Prednisone | |
| | (N = 67) | |
| | All Grades | Grades ≥3 |
| Adverse Reaction | (%) | (%) |

Infections

| | | |
|---|---|---|
| Upper respiratory tract infection^{a} | 39 | 0 |
| Bronchitis | 16 | 0 |
| Pneumonia^{b} | 15 | 7^{#} |

Gastrointestinal disorders

| | | |
|---|---|---|
| Constipation | 37 | 0 |
| Nausea | 36 | 0 |
| Diarrhea | 33 | 3^{#} |
| Vomiting | 21 | 0 |
| Abdominal pain^{c} | 13 | 0 |

General disorders and administration site conditions

| | | |
|---|---|---|
| Fatigue^{d} | 36 | 3 |
| Pyrexia | 34 | 0 |
| Edema peripheral^{e} | 13 | 1^{#} |

Nervous system disorders

| | | |
|---|---|---|
| Peripheral sensory neuropathy | 34 | 1^{#} |
| Dizziness | 10 | 0 |

Respiratory, thoracic and mediastinal disorders

| | | |
|---|---|---|
| Cough^{f} | 24 | 0 |

Psychiatric disorders

| | | |
|---|---|---|
| Insomnia | 22 | 3 |

Musculoskeletal and connective tissue disorders

| | | |
|---|---|---|
| Back pain | 21 | 3^{#} |
| Musculoskeletal chest pain | 12 | 0 |

Metabolism and nutrition disorders

| | | |
|---|---|---|
| Decreased appetite | 15 | 1^{#} |

Skin and subcutaneous tissue disorders

| | | |
|---|---|---|
| Rash | 13 | 0 |
| Pruritus | 12 | 0 |

Vascular disorders

| | | |
|---|---|---|
| Hypertension | 13 | 6^{#} |
| Hypotension | 10 | 3^{#} |

^{a}Upper respiratoiy tract infection includes nasopharyngitis, respiratoiy syncytial virus .nfection, respiratory tract infection, rhinitis, tonsillitis, upper respiratory tract infection, and viral pharyngitis.
^{b}Pneumonia includes lower respiratory tract infection, lung infection, pneumocystis jirovecii pneumonia, pneumonia, and pneumonia bacterial.
^{c}Abdominal pain includes abdominal pain, and abdominal pain upper.
^{d}Fatigue includes asthenia, and fatigue.
^{e}Edema peripheral includes edema, edema peripheral, and peripheral swelling.
^{f}Cough includes cough, and productive cough.
^{#}Only grade 3 adverse reactions occurred.

Clinically relevant adverse reactions in <10% of patients who received DARZALEX FASPRO with bortezomib, melphalan and prednisone (D-VMP) include:
General disorders and administration site conditions: infusion reaction, injection site reaction, chills
Infections: herpes zoster, urinary tract infection, influenza, sepsis
Musculoskeletal and connective tissue disorders: arthralgia, muscle spasms
Nervous system disorders: headache, paresthesia
Metabolism and nutrition disorders: hypocalcemia, hyperglycemia
Respiratory, thoracic and mediastinal disorders: dyspnea, pulmonary edema
Cardiac disorders: atrial fibrillation
   Table 5 summarizes the laboratory abnormalities in patients who received DARZALEX FASPRO with bortezomib, melphalan and prednisone (D-VMP) in PLEIADES.

TABLE 5
Select Hematology Laboratory Abnormalities Worsening from Baseline in
Patients Who Received DARZALEX FASPRO with Bortezomib,
Melphalan and Prednisone (D-VMP) in PLEIADES

| | DARZALEX FASPRO | |
|---|---|---|
| | with Bortezomib, Melphalan and Prednisone ^{a} | |
| | All Grades | Grades 3-4 |
| Laboratory Abnormality | (%) | (%) |
| Decreased leukocytes | 96 | 52 |
| Decreased lymphocytes | 93 | 84 |
| Decreased platelets | 93 | 42 |
| Decreased neutrophils | 88 | 49 |
| Decreased hemoglobin | 48 | 19 |

^{a} Denominator is based on the safety population treated with D-VMP (N = 67).

### Relapsed/Refractory Multiple Myeloma

In Combination with Lenalidomide and Dexamethasone
The safety of DARZALEX FASPRO with lenalidomide and dexamethasone (D-Rd) was evaluated in a single-arm cohort of PLEIADES [see Clinical Studies (14.2)]. Patients received DARZALEX FASPRO 1,800 mg/30,000 units administered subcutaneously once weekly from weeks 1 to 8, once every 2 weeks from weeks 9 to 24 and once every 4 weeks starting with week 25 until disease progression or unacceptable toxicity (N=65) in combination with lenalidomide and dexamethasone. Among these patients, 92% were exposed for 6 months or longer and 20% were exposed for greater than one year.
Serious adverse reactions occurred in 48% of patients who received DARZALEX FASPRO. Serious adverse reactions in >5% of patients included pneumonia, influenza and diarrhea. Fatal adverse reactions occurred in 3.1% of patients.
Permanent discontinuation of DARZALEX FASPRO due to an adverse reaction occurred in 11% of patients who received DARZALEX FASPRO. Adverse reactions resulting in permanent discontinuation of DARZALEX FASPRO in more than 1 patient were pneumonia and anemia.
Dosage interruptions due to an adverse reaction occurred in 63% of patients who received DARZALEX FASPRO. Adverse reactions requiring dosage interruptions in >5% of patients included neutropenia, pneumonia, upper respiratory tract infection, influenza, dyspnea, and blood creatinine increased.
The most common adverse reactions (≥20%) were fatigue, diarrhea, upper respiratory tract infection, muscle spasms, constipation, pyrexia, pneumonia, and dyspnea.
Table 6 summarizes the adverse reactions in patients who received DARZALEX FASPRO with lenalidomide and dexamethasone (D-Rd) in PLEIADES.

TABLE 6
Adverse Reactions (≥10%) in Patients Who Received DARZALEX FASPRO
with Lenalidomide and Dexamethasone (D-Rd) in PLEIADES

| | DARZALEX FASPRO with | |
|---|---|---|
| | Lenalidomide and Dexamethasone | |
| | (N = 65) | |
| | All Grades | Grades ≥3 |
| Adverse Reaction | (%) | (%) |

General disorders and administration site conditions

| | | |
|---|---|---|
| Fattgue^{a} | 52 | 5^{#} |
| Pyrexia | 23 | 2^{#} |
| Edema peripheral | 18 | 3^{#} |

Gastrointestinal disorders

| | | |
|---|---|---|
| Diarrhea | 45 | 5^{#} |
| Constipation | 26 | 2^{#} |
| Nausea | 12 | 0 |
| Vomiting | 11 | 0 |

Infections

| | | |
|---|---|---|
| Upper respiratory tract infection^{b} | 43 | 3^{#} |
| Pneumoni^{c} | 23 | 17 |
| Bronchitis^{d} | 14 | 2^{#} |
| Urinary tract infection | 11 | 0 |

Musculoskeletal and connective tissue disorders

| | | |
|---|---|---|
| Muscle spasms | 31 | 2 |
| Back pain | 14 | 0 |

Respiratory, thoracic and mediastinal disorders

| | | |
|---|---|---|
| Dyspnea^{e} | 22 | 3 |
| Cough^{f} | 14 | 0 |

Nervous system disorders

| | | |
|---|---|---|
| Peripheral sensory neuropathy | 17 | 2^{#} |

Psychiatric disorders

| | | |
|---|---|---|
| Insomnia | 17 | 5^{#} |

Metabolism and nutrition disorders

| | | |
|---|---|---|
| Hyperglycemia | 12 | 9^{#} |
| Hypocalcemia | 11 | 0 |

^{a}Fatigue includes asthenia, and fatigue.
^{b}Upper respiratory tract infection includes nasopharyngitis, pharyngitis, respiratory tract infection viral, rhinitis, sinusitis, upper respiratory tract infection, and upper respiratory tract infection bacterial.
^{c}Pneumonia includes lower respiratory tract infection, lung infection, and pneumonia.
^{d}Bronchitis includes bronchitis, and bronchitis viral.
^{e}Dyspnea includes dyspnea, and dyspnea exertional.
^{f}Cough includes cough, and productive cough.
^{#}Only grade 3 adverse reactions occurred.

Clinically relevant adverse reactions in <10% of patients who received DARZALEX FASPRO with lenalidomide and dexamethasone (D-Rd) include:
Musculoskeletal and connective tissue disorders: arthralgia, musculoskeletal chest pain
Nervous system disorders: dizziness, headache, paresthesia
Skin and subcutaneous tissue disorders: rash, pruritus
Gastrointestinal disorders: abdominal pain
Infections: influenza, sepsis, herpes zoster
Metabolism and nutrition disorders: decreased appetite
Cardiac disorders: atrial fibrillation
General disorders and administration site conditions: chills, infusion reaction, injection site reaction
Vascular disorders: hypotension, hypertension
   Table 7 summarizes the laboratory abnormalities in patients who received DARZALEX FASPRO with lenalidomide and dexamethasone (D-Rd) in PLEIADES.

TABLE 7
Select Hematology Laboratory Abnormalities Worsening from Baseline in
Patients Who Received DARZALEX FASPRO with Lenalidomide and
Dexamethasone (D-Rd) in PLEIADES

| | DARZALEX FASPRO | |
|---|---|---|
| | with Lenalidomide and Dexamethasone ^{a} | |
| Laboratory Abnormality | All Grades (%) | Grades 3-4 (%) |
| Decreased leukocytes | 94 | 34 |
| Decreased lymphocytes | 82 | 58 |
| Decreased platelets | 86 | 9 |
| Decreased neutrophils | 89 | 52 |
| Decreased hemoglobin | 45 | 8 |

^{a} Denominator is based on the safety population treated with D-Rd (N = 65).

### Monotherapy

The safety of DARZALEX FASPRO as monotherapy was evaluated in COLUMBA [see Clinical Trials (14.2)]. Patients received DARZALEX FASPRO 1,800 mg/30,000 units administered subcutaneously or daratumumab 16 mg/kg administered intravenously; each administered once weekly from weeks 1 to 8, once every 2 weeks from weeks 9 to 24 and once every 4 weeks starting with week 25 until disease progression or unacceptable toxicity. Among patients receiving DARZALEX FASPRO, 37% were exposed for 6 months or longer and 1% were exposed for greater than one year.
Serious adverse reactions occurred in 26% of patients who received DARZALEX FASPRO. Fatal adverse reactions occurred in 5% of patients. Fatal adverse reactions occurring in more than 1 patient were general physical health deterioration, septic shock, and respiratory failure.
Permanent discontinuation due to an adverse reaction occurred in 10% of patients who received DARZALEX FASPRO. Adverse reactions resulting in permanent discontinuation of DARZALEX FASPRO in more than 2 patients were thrombocytopenia and hypercalcemia.
Dosage interruptions due to an adverse reaction occurred in 26% of patients who received DARZALEX FASPRO. Adverse reactions requiring dosage interruption in >5% of patients included thrombocytopenia.
The most common adverse reaction (≥20%) was upper respiratory tract infection.
Table 8 summarizes the adverse reactions in COLUMBA.

TABLE 8
Adverse Reactions (≥10%) in Patients Who Received DARZALEX FASPRO or
Intravenous Daratumumab in COLUMBA

| | DARZALEX FASPRO | | Intravenous Daratumumab | |
|---|---|---|---|---|
| | (N = 260) | | (N = 258) | |
| Adverse Reaction | All Grades (%) | Grade ≥3 (%) | All Grades (%) | Grade ≥3 (%) |

Infections

| | | | | |
|---|---|---|---|---|
| Upper respiratory tract infection^{a} | 24 | 1^{#} | 22 | 1^{#} |
| Pneumonia^{b} | 8 | 5 | 10 | 6^{@} |

Gastrointestinal disorders

| | | | | |
|---|---|---|---|---|
| Diarrhea | 15 | 1^{#} | 11 | 0.4^{#} |
| Nausea | 8 | 0.4^{#} | 11 | 0.4^{#} |

General disorders and administration site conditions

| | | | | |
|---|---|---|---|---|
| Fatigue^{c} | 15 | 1^{#} | 16 | 2^{#} |
| Infusion reactions^{d} | 13 | 2^{#} | 34 | 5^{#} |
| Pyrexia | 13 | 0 | 13 | 1^{#} |
| Chills | 6 | 0.4^{#} | 12 | 1^{#} |

Musculoskeletal and connective tissue disorders

| | | | | |
|---|---|---|---|---|
| Back pain | 10 | 2^{#} | 12 | 3^{#} |

Respiratory, thoracic and mediastinal disorders

| | | | | |
|---|---|---|---|---|
| Cough^{e} | 9 | 1^{#} | 14 | 0 |
| Dyspnea^{f} | 6 | 1^{#} | 11 | 1^{#} |

^{a}Upper respiratoiy tract infection includes acute sinusitis, nasophaiyngitis, pharyngitis, respiratoiy syncytial virus infection, respiratcny tract infection, rhinitis, rhinovirus infection, sinusitis, and upper respiratoiy tract infection.
^{b}Pneumonia includes lower respiratoiy tract infection, lung infection, pneumocystis jirovecii pneumonia, and pneumonia.
^{c}Fatigue includes asthenia, and fatigue.
^{d}Infusion reactions includes terms determined by investigators to be related to infusion.
^{e}Cough includes cough, and productive cough.
^{f}Dyspnea includes dyspnea, and dyspnea exertional.
^{#}Only grade 3 adverse reactions occurred.
^{@}Grade 5 adverse reactions occurred.

Clinically relevant adverse reactions in <10% of patients who received DARZALEX FASPRO include:
General disorders and administration site conditions: injection site reaction, peripheral edema
Musculoskeletal and connective tissue disorders: arthralgia, musculoskeletal chest pain, muscle spasms
Gastrointestinal disorders: constipation, vomiting, abdominal pain,
Metabolism and nutrition disorders: decreased appetite, hyperglycemia, hypocalcemia, dehydration
Psychiatric disorders: insomnia
Vascular disorders: hypertension, hypotension
Nervous system disorders: dizziness, peripheral sensory neuropathy, paresthesia
Infections: bronchitis, influenza, urinary tract infection, herpes zoster, sepsis, hepatitis B reactivation
Skin and subcutaneous tissue disorders: pruritus, rash
Cardiac disorders: atrial fibrillation
Respiratory, thoracic and mediastinal disorders: pulmonary edema
   Table 9 summarizes the laboratory abnormalities in COLUMBA.

Select Hematology Laboratory Abnormalities Worsening from
Baseline in Patients Receiving DARZALEX FASPRO or
Intravenous Daratumumab in COLUMBA

| | DARZALEX | | Intravenous | |
|---|---|---|---|---|
| | FASPRO^{a} | | Daratumumab^{a} | |
| | All | Grades | All | Grades |
| | Grades | 3-4 | Grades | 3-4 |
| Laboratory Abnormality | (%) | (%) | (%) | (%) |
| Decreased leukocytes | 65 | 19 | 57 | 14 |
| Decreased lymphocytes | 59 | 36 | 56 | 36 |
| Decreased neutrophils | 55 | 19 | 43 | 11 |
| Decreased platelets | 43 | 16 | 45 | 14 |
| Decreased hemoglobin | 42 | 14 | 39 | 16 |

^{a}Denominator is based on the safety population treated with DARZALEX FASPRO (N = 260) and Intravenous Daratumumab (N = 258).

### 6.2 Immunogenicity

As with all therapeutic proteins, there is the potential for immunogenicity. The detection of antibody formation is highly dependent on the sensitivity and specificity of the assay. Additionally, the observed incidence of antibody (including neutralizing antibody) positivity in an assay may be influenced by several factors including assay methodology, sample handling, timing of sample collection, concomitant medications, and underlying disease. For these reasons, comparison of the incidence of antibodies in the studies described below with the incidence of antibodies in other studies or to other daratumumab products or other hyaluronidase products may be misleading.
Treatment-emergent anti-daratumumab antibodies were tested in 451 patients treated with DARZALEX FASPRO as monotherapy or as part of a combination therapy. One patient (0.2%) who received DARZALEX FASPRO as monotherapy tested positive for anti-daratumumab antibodies and transient neutralizing antibodies. However, the incidence of antibody development might not have been reliably determined because the assays that were used have limitations in detecting anti-daratumumab antibodies in the presence of high concentrations of daratumumab.
Treatment-emergent anti-rHuPH20 antibodies developed in 8% (19/255) of patients who received DARZALEX FASPRO as monotherapy and in 8% (16/192) of patients who received DARZALEX FASPRO as part of a combination therapy. The anti-rHuPH20 antibodies did not appear to affect daratumumab exposures. None of the patients who tested positive for anti-rHuPH20 antibodies tested positive for neutralizing antibodies.

### 6.3 Postmarketing Experience

The following adverse reactions have been identified with use of intravenous daratumumab. Because these reactions are reported voluntarily from a population of uncertain size, it is not always possible to reliably estimate their frequency or establish a causal relationship to drug exposure.
Immune System: Anaphylactic reaction

### Gastrointestinal: Pancreatitis

### 7 Drug Interactions

### 7.1 Effects of Daratumumab on Laboratory Tests

Interference with Indirect Antiglobulin Tests (Indirect Coombs Test)
Daratumumab binds to CD38 on RBCs and interferes with compatibility testing, including antibody screening and cross matching. Daratumumab interference mitigation methods include treating reagent RBCs with dithiothreitol (DTT) to disrupt daratumumab binding [see References (15)] or genotyping. Since the Kell blood group system is also sensitive to DTT treatment, supply K-negative units after ruling out or identifying alloantibodies using DTT-treated RBCs.
If an emergency transfusion is required, administer non-cross-matched ABO/RhD-compatible RBCs per local blood bank practices.
Interference with Serum Protein Electrophoresis and Immunofixation Tests
Daratumumab may be detected on serum protein electrophoresis (SPE) and immunofixation (IFE) assays used for monitoring disease monoclonal immunoglobulins (M protein). False positive SPE and IFE assay results may occur for patients with IgG kappa myeloma protein impacting initial assessment of complete responses by International Myeloma Working Group (IMWG) criteria. In DARZALEX FASPRO-treated patients with persistent very good partial response, where daratumumab interference is suspected, consider using a FDA-approved daratumumab-specific IFE assay to distinguish daratumumab from any remaining endogenous M protein in the patient's serum, to facilitate determination of a complete response.

### 8 Use in Specific Populations

### 8.1 Pregnancy

### Risk Summary

DARZALEX FASPRO can cause fetal harm when administered to a pregnant woman. The assessment of associated risks with daratumumab products is based on the mechanism of action and data from target antigen CD38 knockout animal models (see Data). There are no available data on the use of DARZALEX FASPRO in pregnant women to evaluate drug-associated risk of major birth defects, miscarriage or adverse maternal or fetal outcomes. Animal reproduction studies have not been conducted.
The estimated background risk of major birth defects and miscarriage for the indicated population is unknown. All pregnancies have a background risk of birth defect, loss, or other adverse outcomes. In the U.S. general population, the estimated background risk of major birth defects and miscarriage in clinically recognized pregnancies is 2% to 4% and 15% to 20%, respectively.
The combination of DARZALEX FASPRO and lenalidomide is contraindicated in pregnant women, because lenalidomide may cause birth defects and death of the unborn child. Lenalidomide is only available through a REMS program. Refer to the lenalidomide prescribing information on use during pregnancy.

### Clinical Considerations

### Fetal/Neonatal Adverse Reactions

Immunoglobulin G1 (IgG1) monoclonal antibodies are transferred across the placenta. Based on its mechanism of action, DARZALEX FASPRO may cause depletion of fetal CD38 positive immune cells and decreased bone density. Defer administering live vaccines to neonates and infants exposed to daratumumab in utero until a hematology evaluation is completed.

### Data

### Animal Data

DARZALEX FASPRO for subcutaneous injection contains daratumumab and hyaluronidase. Mice that were genetically modified to eliminate all CD38 expression (CD38 knockout mice) had reduced bone density at birth that recovered by 5 months of age. Data from studies using CD38 knockout animal models also suggest the involvement of CD38 in the regulation of humoral immune responses (mice), feto-maternal immune tolerance (mice), and early embryonic development (frogs).
No systemic exposure of hyaluronidase was detected in monkeys given 22,000 U/kg subcutaneously (12 times higher than the human dose) and there were no effects on embryo-fetal development in pregnant mice given 330,000 U/kg hyaluronidase subcutaneously daily during organogenesis, which is 45 times higher than the human dose.
There were no effects on pre- and post-natal development through sexual maturity in offspring of mice treated daily from implantation through lactation with 990,000 U/kg hyaluronidase subcutaneously, which is 134 times higher than the human doses.

### 8.2 Lactation

### Risk Summary

There is no data on the presence of daratumumab and hyaluronidase in human milk, the effects on the breastfed child, or the effects on milk production. Maternal immunoglobulin G is known to be present in human milk. Published data suggest that antibodies in breast milk do not enter the neonatal and infant circulations in substantial amounts. Because of the potential for serious adverse reactions in the breastfed child when DARZALEX FASPRO is administered with lenalidomide and dexamethasone, advise women not to breastfeed during treatment with DARZALEX FASPRO. Refer to lenalidomide prescribing information for additional information.

### Data

### Animal Data

No systemic exposure of hyaluronidase was detected in monkeys given 22,000 U/kg subcutaneously (12 times higher than the human dose) and there were no effects on post-natal development through sexual maturity in offspring of mice treated daily during lactation with 990,000 U/kg hyaluronidase subcutaneously, which is 134 times higher than the human doses.

### 8.3 Females and Males of Reproductive Potential

DARZALEX FASPRO can Cause Fetal Harm when Administered to a Pregnant Woman [See Use in Specific Populations (8.1)].

### Pregnancy Testing

With the combination of DARZALEX FASPRO with lenalidomide, refer to the lenalidomide labeling for pregnancy testing requirements prior to initiating treatment in females of reproductive potential.

### Contraception

Advise females of reproductive potential to use effective contraception during treatment with DARZALEX FASPRO and for 3 months after the last dose. Additionally, refer to the lenalidomide labeling for additional recommendations for contraception.

### 8.4 Pediatric Use

Safety and effectiveness of DARZALEX FASPRO in pediatric patients have not been established.

### 8.5 Geriatric Use

Of the 291 patients who received DARZALEX FASPRO as monotherapy for relapsed and refractory multiple myeloma, 37% were 65 to <75 years of age, and 19% were 75 years of age or older. No overall differences in effectiveness were observed based on age. Adverse reactions occurring at a higher frequency (≥5% difference) in patients ≥65 years of age included upper respiratory tract infection, urinary tract infection, dizziness, cough, dyspnea, diarrhea, nausea, fatigue, and peripheral edema. Serious adverse reactions occurring at a higher frequency (≥2% difference) in patients ≥65 years of age included pneumonia.
Clinical studies of DARZALEX FASPRO as part of a combination therapy did not include sufficient numbers of patients aged 65 and older to determine whether they respond differently from younger patients.

### 11 Description

Daratumumab is an immunoglobulin G1 kappa (IgG1κ) human monoclonal antibody that binds to the CD38 antigen. Daratumumab is produced in Chinese Hamster Ovary (CHO) cells using recombinant DNA technology. The molecular weight of daratumumab is approximately 148 kDa.
Hyaluronidase (recombinant human) is an endoglycosidase used to increase the dispersion and absorption of co-administered drugs when administered subcutaneously. It is a glycosylated single-chain protein produced by Chinese Hamster Ovary cells containing a DNA plasmid encoding for a soluble fragment of human hyaluronidase (PH20). Hyaluronidase (recombinant human) has a molecular weight of approximately 61 kD.
DARZALEX FASPRO (daratumumab and hyaluronidase-fihj) injection is a sterile, preservative-free, colorless to yellow, and clear to opalescent solution supplied in a single-dose vial for subcutaneous administration.
Each DARZALEX FASPRO 15 mL single-dose vial contains 1,800 mg of daratumumab and 30,000 units of hyaluronidase, L-histidine (4.9 mg), L-histidine hydrochloride monohydrate (18.4 mg), L-methionine (13.5 mg), polysorbate 20 (6 mg), sorbitol (735.1 mg), and Water for Injection, USP.

### 12 Clinical Pharmacology

### 12.1 Mechanism of Action

CD38 is a transmembrane glycoprotein (48 kDa) expressed on the surface of hematopoietic cells, including multiple myeloma and other cell types and tissues and has multiple functions, such as receptor mediated adhesion, signaling, and modulation of cyclase and hydrolase activity. Daratumumab is an IgG1κ human monoclonal antibody (mAb) that binds to CD38 and inhibits the growth of CD38 expressing tumor cells by inducing apoptosis directly through Fc mediated cross linking as well as by immune-mediated tumor cell lysis through complement dependent cytotoxicity (CDC), antibody dependent cell mediated cytotoxicity (ADCC) and antibody dependent cellular phagocytosis (ADCP). A subset of myeloid derived suppressor cells (CD38+MDSCs), regulatory T cells (CD38+T_{regs}) and B cells (CD38+B_{regs}) are decreased by daratumumab.
Hyaluronan is a polysaccharide found in the extracellular matrix of the subcutaneous tissue. It is depolymerized by the naturally occurring enzyme hyaluronidase. Unlike the stable structural components of the interstitial matrix, hyaluronan has a half-life of approximately 0.5 days. Hyaluronidase increases permeability of the subcutaneous tissue by depolymerizing hyaluronan. In the doses administered, hyaluronidase in DARZALEX FASPRO acts locally. The effects of hyaluronidase are reversible and permeability of the subcutaneous tissue is restored within 24 to 48 hours.

### 12.2 Pharmacodynamics

NK cells express CD38 and are susceptible to daratumumab mediated cell lysis. Decreases in absolute counts and percentages of total NK cells (CD16+CD56+) and activated (CD16+CD56^{dim}) NK cells in peripheral whole blood and bone marrow were observed with DARZALEX FASPRO treatment.

### Cardiac Electrophysiology

DARZALEX FASPRO as a large protein has a low likelihood of direct ion channel interactions. There is no evidence from non-clinical or clinical data to suggest that DARZALEX FASPRO has the potential to delay ventricular repolarization.

### Exposure-Response Relationship

The exposure-response relationship and time course of pharmacodynamics of DARZALEX FASPRO have not been fully characterized.

### 12.3 Pharmacokinetics

Following the administration of the recommended dose of DARZALEX FASPRO 1,800 mg/30,000 units (1,800 mg daratumumab and 30,000 units hyaluronidase) subcutaneously once weekly for 8 weeks, the mean±standard deviation (SD) maximum trough concentrations (C_{trough} following the 8^{th} dose) were 593±306 μg/mL compared to 522±226 μg/mL for daratumumab 16 mg/kg administered intravenously, with a geometric mean ratio of 108% (90% CI: 96, 122). The estimated median daratumumab area under the concentration-time curves (AUC_{0-7 days}) and daratumumab peak concentration (Cₘₐₓ) following the 8^{th} dose were comparable between DARZALEX FASPRO and intravenous daratumumab (4017 μg/mL·day vs. 4,019 μg/mL·day for AUC_{0-7 days} and 592 μg/mL vs. 688 μg/mL for Cₘₐₓ).
Following the recommended dose of DARZALEX FASPRO 1,800 mg/30,000 units, Cₘₐₓ increased 4.8-fold and AUC_{0-7 days} increased 5.4-fold from the 1s^{t} dose to the 8^{th} dose.

### Absorption

At the recommended dose of DARZALEX FASPRO 1,800 mg/30,000 units, the absolute bioavailability is 69%, with peak concentrations occurring around 3 days (Tₘₐₓ).

### Distribution

The estimated mean (coefficient of variation, CV) volume of distribution for the central compartment is 5.2 L (37%) and peripheral compartment was 3.8 L.

### Elimination

Daratumumab is cleared by parallel linear and nonlinear saturable target mediated clearances. The estimated mean (CV %) linear clearance of daratumumab is 119 mL/day. The estimated mean (CV %) elimination half-life associated with linear clearance is 20 days (22%).

### Specific Populations

The following population characteristics have no clinically meaningful effect on the pharmacokinetics of daratumumab in patients administered DARZALEX FASPRO as monotherapy or as combination therapy: sex, age (33 to 92 years), race, renal impairment [Creatinine clearance (CLcr) 15 to 89 mL/min as determined by the Cockcroft-Gault formula], and mild hepatic impairment (total bilirubin 1 to 1.5 times ULN and AST>ULN). The effect of moderate and severe hepatic impairment on daratumumab pharmacokinetics is unknown.

### Body Weight

After administration of DARZALEX FASPRO 1,800 mg/30,000 units as monotherapy, the mean maximum C_{trough} after the 8^{th} dose was 12% lower in the higher body weight (BW) group (>85 kg) while the mean maximum C_{trough} was 81% higher in the lower BW group (≤50 kg) compared to the corresponding BW groups in the intravenous daratumumab arm.

### 13 Nonclinical Toxicology

### 13.1 Carcinogenesis, Mutagenesis, Impairment of Fertility

No carcinogenicity or genotoxicity studies have been conducted with daratumumab. No animal studies have been performed to evaluate the potential effects of daratumumab on reproduction or development, or to determine potential effects on fertility in males or females.

No carcinogenicity, genotoxicity, or fertility studies were conducted for recombinant human hyaluronidase. There were no effects on reproductive tissues and function and no systemic exposure of hyaluronidase in monkeys given 22,000 U/kg/week subcutaneously (12 times higher than the human dose) for 39 weeks. As hyaluronidase is a recombinant form of the endogenous human hyaluronidase, no carcinogenicity, mutagenesis, or effects on fertility are expected.

### 14 Clinical Studies

### 14.1 Newly Diagnosed Multiple Myeloma

In Combination with Bortezomib, Melphalan and Prednisone
The efficacy of DARZALEX FASPRO with bortezomib, melphalan and prednisone (D-VMP) was evaluated in a single-arm cohort of PLEIADES (NCT03412565), a multi-cohort, open-label trial. Eligible patients were required to have newly diagnosed multiple myeloma who are ineligible for transplant. Patients received DARZALEX FASPRO 1,800 mg/30,000 units administered subcutaneously once weekly from weeks 1 to 6, once every 3 weeks from weeks 7 to 54 and once every 4 weeks starting with week 55 until disease progression or unacceptable toxicity; bortezomib 1.3 mg/m² subcutaneously twice weekly on Weeks 1, 2, 4 and 5 for the first 6-week cycle (Cycle 1; 8 doses), followed by once weekly on Weeks 1, 2, 4 and 5 for eight more 6-week cycles (Cycles 2-9; 4 doses per cycle); and melphalan 9 mg/m² and prednisone 60 mg/m² orally on Days 1 to 4 of the nine 6-week cycles (Cycles 1-9). The major efficacy outcome measure was overall response rate (ORR).
A total of 67 patients received DARZALEX FASPRO with VMP. The median age was 75 years (range: 66 to 86); 46% were male; 69% were White, 8% Asian, and 2% Black or African American; and 33% had ISS Stage I, 45% had ISS Stage II, and 22% had ISS Stage III disease.
Efficacy results are summarized in Table 10

TABLE 10
Efficacy Results from PLEIADES in Patients Who Received D-VMP

| | D-VMP |
|---|---|
| | (N = 67) |
| Overall response rate (sCR+CR+VGPR+PR), n | 59 (88%) |
| (%)a | |
| 95% CI (%) | (78%, 95%) |
| Stringent complete response (sCR) | 5 (8%) |
| Complete response (CR) | 7 (10%) |
| Very good partial response (VGPR) | 31 (46%) |
| Partial response (PR) | 16 (24%) |

CI = confidence interval
^{a}Based on treated patients

### 14.2 Relapsed/Refractory Multiple Myeloma

In Combination with Lenalidomide and Dexamethasone
The efficacy of DARZALEX FASPRO with lenalidomide and dexamethasone (D-Rd) was evaluated in a single-arm cohort of PLEIADES (NCT03412565), a multi-cohort, open-label trial. Patients received DARZALEX FASPRO 1,800 mg/30,000 units administered subcutaneously once weekly from weeks 1 to 8, once every 2 weeks from weeks 9 to 24 and once every 4 weeks starting with week 25 until disease progression or unacceptable toxicity with lenalidomide 25 mg once daily orally on Days 1-21 of each 28-day cycle; and dexamethasone 40 mg per week (or a reduced dose of 20 mg per week for patients >75 years or BMI <18.5). The major efficacy outcome measure was ORR.
A total of 65 patients received DARZALEX FASPRO with Rd. The median age was 69 years (range: 33 to 82); 69% were male; 69% were White, and 3% Black or African American; and 42% had ISS Stage I, 30% had ISS Stage II, and 28% had ISS Stage III disease. Patients had received a median of 1 prior line of therapy. A total of 52% of patients had a prior ASCT; 95% of patients received a prior PI; 59% received a prior immunomodulatory agent, including 22% who received prior lenalidomide; and 54% of patients received both a prior PI and immunomodulatory agent.
Efficacy results are summarized in Table 11.

TABLE 11
Efficacy Results from PLEIADES in Patients Who Received D-Rd

| | D-Rd |
|---|---|
| | (N = 65) |
| Overall response rate (sCR+CR+VGPR+PR), n (%)^{a} | 59 (91%) |
| 95% CI (%) | (81%, 97%) |
| Stringent complete response (sCR) | 4 (6%) |
| Complete response (CR) | 8 (12%) |
| Very good partial response (VGPR) | 30 (46%) |
| Partial response (PR) | 17 (26%) |

CI = confidence interval
^{a}Based on treated patients

### Monotherapy

The efficacy of DARZALEX FASPRO as monotherapy was evaluated in COLUMBA (NCT03277105), an open-label, randomized, non-inferiority study. Eligible patients were required to have relapsed or refractory multiple myeloma who had received at least 3 prior lines of therapy including a proteasome inhibitor and an immunomodulatory agent or who were double-refractory to a proteasome inhibitor and an immunomodulatory agent. Patients were randomized to receive DARZALEX FASPRO (1,800 mg/30,000 units) administered subcutaneously or daratumumab 16 mg/kg administered intravenously; each administered once weekly from weeks 1 to 8, once every 2 weeks from weeks 9 to 24 and once every 4 weeks starting with week 25 until unacceptable toxicity or disease progression. The major efficacy outcome measures were ORR by the IMWG response criteria and maximum C_{trough} at pre-dose Cycle 3 Day 1 [see Clinical Pharmacology (12.3)]. Randomization was stratified by body weight, myeloma type, and number of prior lines of therapy.
A total of 522 patients were randomized: 263 to the DARZALEX FASPRO arm and 259 to the intravenous daratumumab arm. The median age was 67 years (range: 33 to 92); 55% were male; and 78% were White, 14% Asian, and 3% Black or African American. The median weight was 73 kg (range: 29 to 138). Patients had received a median of 4 prior lines of therapy. A total of 51% of patients had a prior ASCT; 100% of patients received both a PI and an immunomodulatory agent. Forty-nine percent of patients were refractory both a PI and an immunomodulatory agent. Eighty-two percent of patients were refractory to their last line of prior systemic therapy.
The results show that DARZALEX FASPRO 1,800 mg/30,000 units administered subcutaneously is non-inferior to daratumumab 16 mg/kg administered intravenously in terms of ORR and maximum trough concentration [see Clinical Pharmacology (12.3)]. Median progression-free survival was 5.6 months in the DARZALEX FASPRO arm and 6.1 months in the intravenous daratumumab arm. ORR results are provided in Table 12.

TABLE 12
Efficacy Results from COLUMBA

| | | Intravenous |
|---|---|---|
| | DARZALEX | Daratumumab |
| | FASPRO (N = 263) | (N = 259) |
| Overall response (sCR+30CR+ | 108 (41%) | 96 (37%) |
| 30VGPR+30PR), n (%)^{a} | | |
| 95% CI (%) | (35%, 47%) | (31%, 43%) |
| Ratio of response rates (95% CI)^{b} | | 1.11 (0.89, 1.37) |
| CR or better, n (%) | 5 (1.9%) | 7 (2.7%) |
| Very good partial response (VGPR) | 45 (17%) | 37 (14%) |
| Partial response (PR) | 58 (22%) | 52 (20%) |

^{a}Based on intent-to-treat population.

### 15 References

1. Chapuy, C I, R T Nicholson, M D Aguad, et al., 2015, Resolving the daratumumab interference with blood compatibility testing, Transfusion, 55:1545-1554 (accessible at http://onlinelibrary.wiley.com/doi/10.1111/trf.13069/epdf).

### 16 how Supplied/Storage and Handling

DARZALEX FASPRO (daratumumab and hyaluronidase-fihj) injection is a sterile, preservative-free, colorless to yellow, and clear to opalescent solution for subcutaneous use supplied as individually packaged single-dose vials providing 1,800 mg of daratumumab and 30,000 units of hyaluronidase per 15 mL (NDC 57894-503-01).
Store DARZALEX FASPRO vials in a refrigerator at 2° C. to 8° C. (36° F. to 46° F.) in the original carton to protect from light.
Do not freeze or shake.

### 17 Patient Counseling Information

Advise the patient to read the FDA-approved patient labeling (Patient Information).

### Hypersensitivity and Other Administration Reactions

Advise patients to seek immediate medical attention for any of the following signs and symptoms of systemic administration-related reactions: itchy, runny or blocked nose; chills, nausea, throat irritation, cough, headache, shortness of breath or difficulty breathing [see Warnings and Precautions (5.1)].

### Neutropenia

Advise patients to contact their healthcare provider if they have a fever [see Warnings and Precautions (5.2)].

### Thrombocytopenia

Advise patients to contact their healthcare provider if they have bruising or bleeding [see Warnings and Precautions (5.3)].

### Embryo-Fetal Toxicity

Advise pregnant women of the potential hazard to a fetus. Advise females of reproductive potential to inform their healthcare provider of a known or suspected pregnancy [see Warnings and Precautions (5.4), Use in Specific Populations (8.1, 8.3)].
Advise females of reproductive potential to avoid becoming pregnant during treatment with DARZALEX FASPRO and for at least 3 months after the last dose [see Use in Specific Populations (8.1, 8.3)].
Advise patients that lenalidomide has the potential to cause fetal harm and has specific requirements regarding contraception, pregnancy testing, blood and sperm donation, and transmission in sperm. Lenalidomide is only available through a REMS program [see Use in Specific Populations (8.1, 8.3)].
Interference with Laboratory Tests
Advise patients to inform their healthcare provider, including personnel at blood transfusion centers, that they are taking DARZALEX FASPRO, in the event of a planned transfusion [see Warnings and Precautions (5.5)].
Advise patients that DARZALEX FASPRO can affect the results of some tests used to determine complete response in some patients and additional tests may be needed to evaluate response [see Warnings and Precautions (5.6)].

### Hepatitis B Virus (HBV) Reactivation

Advise patients to inform healthcare providers if they have ever had or might have a hepatitis B infection and that DARZALEX FASPRO could cause hepatitis B virus to become active again [see Adverse Reactions (6.1)].

The examples and embodiments described herein are for illustrative purposes only and various modifications or changes suggested to persons skilled in the art are to be included within the spirit and purview of this application and scope of the appended claims.

We claim:

1. A method of treating multiple myeloma comprising administering an approved drug product containing daratumumab and hyaluronidase to an adult patient with multiple myeloma in an amount that is described in a drug product label for the drug product.

2. The method of claim 1, wherein the multiple myeloma is newly diagnosed in patients who are ineligible for autologous stem cell transplant.

3. The method of claim 2, wherein the drug product is administered in combination with bortezomib, melphalan and prednisone.

4. The method of claim 2, wherein the drug product is administered in combination with lenalidomide and dexamethasone.

5. The method of claim 1, wherein the multiple myeloma is relapsed or refractory to at least one prior line of therapy.

6. The method of claim 5, wherein the drug product is administered in combination with lenalidomide and dexamethasone.

7. The method of claim 1, wherein the multiple myeloma is relapsed or refractory to at least three prior lines of therapy.

8. The method of claim 7, wherein the three prior lines of therapy include a proteasome inhibitor or an immunomodulatory agent.

9. The method of claim 1, wherein the multiple myeloma is double-refractory to a proteasome inhibitor and an immunomodulatory agent.

10. The method of any one of claims 3 to 9, wherein administration of daratumumab and hyaluronidase provides an increase in the overall response rate (ORR) of the adult patient with multiple myeloma.

11. The method of any one of the preceding claims, wherein the daratumumab and hyaluronidase are administered subcutaneously at a dose of about 1800 mg daratumumab and 30,000 units of hyaluronidase.

12. The method of claim 11, wherein the daratumumab and hyaluronidase are administered weekly for 8 weeks, followed by administration every two weeks for eight weeks, followed by administration every 4 weeks until disease progression.

13. A method of selling an approved drug product comprising daratumumab and hyaluronidase, the method comprising selling such drug product, wherein a drug product label for a reference listed drug for such drug product includes instructions for treating multiple myeloma.

14. The method of claim 13, wherein the drug product is a biosimilar drug product, a Biologic License Application drug product or a supplemental Biologic License Application drug product.

15. A method of offering for sale an approved drug product comprising daratumumab and hyaluronidase, the method comprising offering for sale such drug product, wherein a drug product label for a reference listed drug for such drug product includes instructions for treating multiple myeloma.

16. The method of claim 15, wherein the drug product is a biosimilar drug product, a Biologic License Application drug product or a supplemental Biologic License Application drug product.

17. A method of selling an approved drug product comprising daratumumab and hyaluronidase, the method comprising selling such drug product, wherein the drug product label for a reference listed drug for such drug product comprises ORR data.

18. A method of offering for sale an approved drug product comprising daratumumab and hyaluronidase, said method comprising offering for sale such drug product, wherein the drug product label for a reference listed drug for such drug product comprises ORR data.

19. A method of improving ORR in an adult patient with multiple myeloma, the method comprising administering to the adult patient an approved drug product comprising daratumumab and hyaluronidase.

20. The method of claim 17, 18 or 19, wherein the approved drug product is a biosimilar drug product, a Biologic License Application drug product or a supplemental Biologic License Application drug product.


SEQUENCE LISTING (22 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 14 aa, Homo sapiens
SKRNIQFSCKNIYR

SEQ ID NO: 3 - protein, 14 aa, Homo sapiens
EKVQTLEAWVIHGG

SEQ ID NO: 4 - protein, 122 aa, Homo sapiens [sequence omitted]

SEQ ID NO: 5 - protein, 107 aa, Homo sapiens [sequence omitted]

SEQ ID NO: 6 - protein, 5 aa, Homo sapiens
SFAMS

SEQ ID NO: 7 - protein, 17 aa, Homo sapiens
AISGSGGGTYYADSVKG

SEQ ID NO: 8 - protein, 17 aa, Homo sapiens
AISGSGGGTYYADSVKG

SEQ ID NO: 9 - protein, 11 aa, Homo sapiens
RASQSVSSYLA

SEQ ID NO: 10 - protein, 7 aa, Homo sapiens
DASNRAT

SEQ ID NO: 11 - protein, 10 aa, Homo sapiens
QQRSNWPPTF

SEQ ID NO: 12 - protein, 452 aa, Homo sapiens
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, Homo sapiens
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, Homo sapiens
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, Homo sapiens
EIVLTQSPATLSLSPGERATLSCRASQSVSSYLAWYQQKPGQAPGLLIYDASNRASGIPARFSGSGSGTDFTLTISSLEPEDFAVYYCQQRSNWPLTFGGGTKVEIK

SEQ ID NO: 18 - protein, 120 aa, Homo sapiens
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, Homo sapiens
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, Mus Musculus [sequence omitted]

SEQ ID NO: 21 - protein, 107 aa, Mus Musculus [sequence omitted]

SEQ ID NO: 22 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL